(19) United States
(12) Patent Application Publication
(10) Pub. No.: US 2004/0265368 A1
(43) Pub. Date: Dec. 30, 2004

(54) Combination compositions of camptothecins and fluoropyrimidines

(76) Inventors: Lawrence Mayer, North Vancouver (CA); Marcel Bally, Bowen Island (CA); Murray Webb, North Vancouver (CA); Paul Tardi, Surrey (CA); Sharon Johnstone, Vancouver (CA)

Correspondence Address: MORRISON & FOERSTER LLP, 3811 VALLEY CENTRE DRIVE, SUITE 500, SAN DIEGO, CA, 92130-2332, US

(21) Appl. No.: 10/817,735

(22) Filed: Apr. 2, 2004

Related U.S. Application Data
(60) Provisional application No. 60/460,169, filed on Apr. 2, 2003.

(51) Int. Cl. A61K 31/4745; A61K 9/127

(52) U.S. Cl. 424/450; 514/283; 514/50; 514/251

(57) ABSTRACT

Compositions which comprise liposomes having stably associated therewith a camptothecin and a fluoropyrimidine are useful in achieving enhanced therapeutic effects when combinations of these drugs are administered.

### CROSS-REFERENCE TO RELATED APPLICATIONS

This application claims benefit under 35 U.S.C. § 119(e) of U.S. Ser. No. 60/460,169 filed 2 Apr. 2003, the contents of which are incorporated herein by reference.

### TECHNICAL FIELD

The invention relates to compositions and methods for improved delivery of combinations of therapeutic agents. More particularly, the invention concerns delivery systems which provide combinations of pyrimidine and camptothecin agents and derivatives thereof.

### BACKGROUND ART

The progression of many life-threatening diseases such as cancer, AIDS, infectious diseases, immune disorders and cardiovascular disorders are influenced by multiple molecular mechanisms. Due to this complexity, achieving cures with a single agent has been met with limited success. Thus, combinations of agents have often been used to combat disease, particularly in the treatment of cancers. It appears that there is a strong correlation between the number of agents administered and cure rates for cancers such as acute lymphocytic leukemia and metastatic colorectal cancer (Frei, et al., Clin. Cancer Res. (1998) 4:2027-2037; Fisher, M. D., Clin Colorectal Cancer (2001) Aug; 1(2):85-86). In particular, combinations of pyrimidine analogs and camptothecin derivatives have been used to successfully treat gastric and colorectal cancers and are being investigated for their effects on esophageal cancers. Clinical trials utilizing the pyrimidine analog, fluorouracil (5-FU), with a camptothecin (irinotecan) and leucovorin demonstrated enhanced survival over 5-FU treatment alone for advanced colorectal cancer (Fisher, M. D. (supra); and U.S. Pat. No. 6,403,569). Fisher also showed that patients who had developed 5-FU-resistant colorectal cancers after a first-line treatment of 5-FU also exhibited increased survival with a combination of 5-FU, irinotecan (CPT-11) and leucovorin, thus demonstrating another potential use of these drug combinations in overcoming drug-resistant cancers.

Camptothecin is a quinoline-based alkaloid found in the bark of the Chinese camptotheca tree and the Asian nothapodytes tree. Many derivatives of Camptothecin including semi-synthetic or synthetic derivatives, such as topotecan and irinotecan, have a unique ability to inhibit Topoisomerase I which has made them highly active cell-killing agents. Topoisomerase I is a cellular enzyme responsible for the winding and unwinding of DNA. If the DNA cannot be unwound then transcription of the DNA message cannot occur and protein will not be synthesized, resulting in the eventual death of the cell. Cells that are dividing at a rapid rate, such as cancer cells, are particularly sensitive to camptothecin derivatives as their DNA is constantly being unwound in order to be replicated for daughter cells. In the open state, the DNA is vulnerable to insertion of camptothecin drugs which has been shown to result in the eventual breaking of the DNA and cell death.

Pyrimidine analogs, such as 5-FU and cytarabine (cytosine arabinoside or araC), are antimetabolites that resemble pyrimidine nucleotides. Most antimetabolites have different modes of action. For example, 5-FU acts as a suicide inactivator of thymidylate synthase, covalently modifying the enzyme's active site. Thymidylate synthase, which is the limiting irreversible step in de novo synthesis of DNA, catalyzes the conversion of dUMP to dTMP. Temporary blockage of this step results in cell death. In contrast, cytarabine is bioactivated to araCMP by cellular enzymes which allows it to compete with CTP as an alternate substrate for DNA polymerase. The araCMP incorporates into the DNA therefore inhibiting further synthesis of the growing DNA strand. Such antiproliferative properties have enabled antimetabolites to effectively treat cancers of the colon, breast, head, neck stomach and pancreas as rapidly dividing cells are highly susceptible to the effects of these drugs.

Although 5-FU was introduced into clinical trials approximately 40 years ago, it was not until the early 1990s that trials involving combinations of camptothecin derivatives with pyrimidine analogs were investigated (Furuta, T., and Yokokura, T., Gan To Kagaku Ryoho (1991) Mar; 18(3):393-402). For many years, researchers went on to demonstrate promising improvements in cancer treatment by administering free drug cocktails of a number of pyrimidine/camptothecin combinations (see PCT patent application Nos. WO 00/66125 and WO 01/62235). Despite the advantages associated with the use of pyrimidine/camptothecin drug cocktails, there are various drawbacks that limit their therapeutic use. For instance, administration of free drug cocktails often results in rapid clearance of one or all of the drugs before reaching the tumor site. For this reason, many drugs have been incorporated into delivery vehicles designed to ‘shield’ them from mechanisms that would otherwise result in their clearance from the bloodstream.

It is well known that liposomes have the ability to provide this ‘shielding’ effect and they are thus able to extend the half-life of therapeutic agents. Encapsulation into well-designed delivery vehicles can also result in coordinated pharmacokinetics of encapsulated drugs. However, formulation of specific drugs or more than one drug into delivery vehicles has proven to be difficult because the lipid composition of the vehicle often differentially affects the pharmacokinetics of individual drugs. Thus a composition that is suitable for retention and release of one drug may not be suitable for the retention and release of a second drug. Presently, although there are a number of active pyrimidine/camptothecin drug combinations being successfully utilized in clinical trials, a pharmaceutical preparation designed to control the pharmacokinetics, and thus tumor delivery, of both drugs has not been described. For example, U.S. Pat. No. 6,403,569 issued Jun. 11, 2002, to W. Achterrath, claims a method for treating cancer by administering a synergistic amount of a camptothecin derivative, 5-FU, and leucovorin (a compound related to the vitamin folic acid which is a standard practice of care during 5-FU treatment) providing that there is at least 200 mg/m² of leucovorin, yet no pharmaceutical preparations designed to ensure delivery and/or enhance circulation lifetimes of these drugs were suggested.

Investigators of the present invention have identified particular delivery vehicle formulations required to accommodate a combination of pyrimidine and camptothecin derivatives, and which result in superior drug loading and sustained drug release of each agent. They have further demonstrated that synergistic ratios of these drugs, when encapsulated in liposomes, can be maintained over time in the blood compartment resulting in enhanced efficacy compared to the free drug cocktail.

### DISCLOSURE OF THE INVENTION

The invention relates to compositions and methods for administering effective amounts of fluoropyrimidine/camptothecin drug combinations using liposomal vehicles that are stably associated with at least one fluoropyrimidine and one water-soluble camptothecin. These compositions allow the two or more agents to be delivered to the disease site in a coordinated fashion, thereby assuring that the agents will be present at the disease site at a desired ratio. This result will be achieved whether the agents are co-encapsulated in lipid-based delivery vehicles, or are separately encapsulated in a single lipid-based delivery vehicle administered such that desired ratios are maintained at the disease site. The pharmacokinetics (PK) of the composition are controlled by the lipid-based delivery vehicles themselves such that coordinated delivery is achieved (provided that the PK of the delivery systems are comparable).

Thus, in one aspect, the invention provides a liposome composition for parenteral administration comprising at least one fluoropyrimidine and one water-soluble camptothecin associated with the liposomes at therapeutically effective ratios that are able to exert a desired, preferably non-antagonistic effect. The therapeutically effective ratio of the agents is determined by assessing the biological activity or effects of the agents on relevant cell culture or cell-free systems, as well as tumor cell homogenates from individual patient biopsies, over a range of concentrations. Preferred combinations are irinotecan and fluorouracil (5-FU) or irinotecan and fluorodeoxyuridine (FUDR). Any method which results in determination of a ratio of agents which maintains a desired therapeutic effect may be used.

The composition comprises at least one fluoropyrimidine and one water-soluble camptothecin in a mole ratio of the fluoropyrimidine to the water-soluble camptothecin which exhibits a desired, preferably non-antagonistic biologic effect to relevant cells in culture or cell-free systems and tumor cell homogenates. By “relevant” cells, applicants refer to at least one cell culture or cell line which is appropriate for testing the desired biological effect. As these agents are used as antineoplastic agents, “relevant” cells are those of cell lines identified by the Developmental Therapeutics Program (DTP) of the National Cancer Institute (NCI)/National Institutes of Health (NIH) as useful in their anticancer drug discovery program. Currently the DTP screen utilizes 60 different human tumor cell lines. The desired activity on at least one of such cell lines would need to be demonstrated. By “tumor cell homogenate”, the applicant refers to cells generated from the mechanical or chemical disruption of patient biopsies or tumors into whole cell samples. Extraction of whole tumors or tumor biopsies can be achieved through standard medical techniques by a qualified physician and homogenization of the tissue into single, whole cells can be carried out in the laboratory using a number of methods well-known in the art.

In another aspect, the invention is directed to a method to deliver a therapeutically effective amount of a fluoropyrimidine/water-soluble camptothecin combination to a desired target by administering the compositions of the invention.

The invention is also directed to a method to deliver a therapeutically effective amount of a fluoropyrimidine/water-soluble camptothecin drug combination by administering a fluoropyrimidine stably associated with a first delivery vehicle and a water-soluble camptothecin stably associated with a second delivery vehicle. The first and second delivery vehicles may be contained in separate vials, the contents of the vials being administered to a patient simultaneously or sequentially. In one embodiment, the ratio of the fluoropyrimidine and the water-soluble camptothecin administered is non-antagonistic.

In another aspect of the invention, leucovorin (a compound related to folic acid) is administered with compositions of the invention in order to stabilize the fluoropyrimidines in vivo.

In another aspect, the invention is directed to a method to prepare a therapeutic composition comprising liposomes containing a ratio of at least one fluoropyrimidine and one water-soluble camptothecin which provides a desired therapeutic effect which method comprises providing a panel of at least one fluoropyrimidine and one water-soluble camptothecin wherein the panel comprises at least one, but preferably a multiplicity of ratios of said drugs, testing the ability of the members of the panel to exert a biological effect on a relevant cell culture or cell-free systems and tumor cell homogenate over a range of concentrations, selecting a member of the panel wherein the ratio provides a desired therapeutic effect on said cell culture or cell-free system over a suitable range of concentrations; and stably associating the ratio of drugs represented by the successful member of the panel into lipid-based drug delivery vehicles. In preferred embodiments, the above-mentioned desired therapeutic effect is non-antagonistic.

As further described below, in a preferred embodiment, in designing an appropriate combination in accordance with the method described above, the non-antagonistic ratios are selected as those that have a combination index (CI) of ≦1.1. In further embodiments, suitable liposomal formulations are designed such that they stably incorporate an effective amount of a fluoropyrimidine/water-soluble camptothecin combination and allow for the sustained release of both drugs in vivo. Preferred formulations contain at least one negatively charged lipid, such as phosphatidylglycerol and contain at least one sterol, such as cholesterol.

### BRIEF DESCRIPTION OF THE DRAWINGS

FIG. 1 is a graph showing irinotecan (CPT-11) loading over time at 50° C. into DSPC/DSPG/Chol (70:20:10 mole ratio) liposomes containing 100 mM Cu(gluconate)₂, 220 mM TEA, pH 7.4 and passively entrapped FUDR.

FIG. 2A is a graph comparing CPT-11 loading over time at 50° C. into DSPC/DSPG/Chol (70:20:10 mole ratio) liposomes containing either 100 mM copper gluconate, 220 mM TEA, pH 7.4 or 100 mM CuSO₄, 265 mM TEA, pH 7.4; and passively entrapped FUDR.

FIG. 2B is a graph comparing FUDR retention over time (after CPT-11 loading) in DSPC/DSPG/Chol (70:20:10 mole ratio) liposomes containing either 100 mM copper gluconate, 220 mM TEA, pH 7.4 or 100 mM CuSO₄, 265 mM TEA, pH 7.4; and passively entrapped FUDR with HBS, pH 7.4 as the external buffer solution.

FIG. 3 is a graph comparing CPT-11 loading over time into DSPC/DSPG/Chol (70:20:10 mole ratio) liposomes containing either 100 mM copper gluconate, 220 mM TEA, pH 7.4 or 150 mM copper tartrate, 20 mM Hepes, pH 7.4; and passively entrapped FUDR with HBS, pH 7.4 as the external buffer solution.

FIG. 4A is a graph comparing CPT-11 retention over time in DSPC/DSPG/Chol liposomes loaded with CPT-11 and FUDR as the amount of liposomal-cholesterol is increased from 5 mole % to 20 mole %. FUDR was passively entrapped into liposomes containing 250 mM CuSO₄ and CPT-11 was then actively loaded prior to administration and measurement of CPT-11 levels.

FIG. 4B is a graph comparing FUDR retention over time in DSPC/DSPG/Chol liposomes loaded with CPT-11 and FUDR as the amount of liposomal-cholesterol is increased from 5 mole % to 20 mole %. FUDR was passively entrapped into liposomes containing 250 mM CuSO₄ and CPT-11 was then actively loaded prior to administration and measurement of FUDR levels.

FIG. 5A is a graph showing the combination index (CI) plotted as a function of the fraction of HT-29 human colorectal cells affected by combinations of FUDR:CPT-11 at various mole ratios: 10:1 (solid squares); 5:1 (solid circles); 1:1 (solid triangles); 1:5 (solid inverted triangles); and 1:10 (open circles).

FIG. 5B is a graph showing the combination index (CI) plotted as a function of the fraction of H460 human large cell carcinoma cells affected by combinations of FUDR:CPT-11 at various mole ratios: 10:1 (solid squares); 5:1 (solid circles); 1:1 (solid triangles); 1:5 (solid inverted triangles); and 1:10 (open circles).

FIG. 5C is a graph showing the combination index (CI) plotted as a function of the fraction of HCT-116 human colorectal carcinoma cells affected by combinations of FUDR:CPT-11 at various mole ratios: 10:1 (solid squares); 5:1 (solid circles); 1:1 (solid triangles); 1:5 (solid inverted triangles); and 1:10 (open circles).

FIG. 5D is a graph showing the compiled data sets from various tumor types plotted as a function of their relative synergy values from FUDR:CPT-11 at mole ratios of 1:5, 1:1, and 1:10.

FIG. 6A is a graph of the CPT-11/FUDR ratio (mol/mol) in the plasma as a function of time after intravenous administration of CPT-11 and FUDR in dual-loaded liposomes and as a free drug cocktail to CD-11 mice.

FIG. 6B is a graph of the CPT-11/FUDR ratio (mol/mol) in the plasma as a function of time after intravenous administration of CPT-11/FUDR dual-loaded liposome to SCID-Rag2M mice.

FIG. 7 is a graph showing the simultaneous encapsulation of CPT-11 (closed circles) and FUDR (open circles) into DSPC/DSPG/Chol (7:2:1) liposomes containing copper gluconate. Both drugs were loaded by heating the drug mixture in the presence of the liposomes at 50° C. Loading was monitored over a 2 hour time course.

FIG. 8A is a graph of tumor weight versus time after tumor cell inoculation with human HT-29 colon adenocarcinoma cells followed by administration of saline (control; solid circles), a free drug cocktail of CPT-11:FUDR at a 1:1 synergistic ratio (open squares, and solid triangle), and a liposomal formulation of CPT-11:FUDR at a 1:1 synergistic ratio (open inverted triangles).

FIG. 8B is a graph of tumor weight versus time after tumor cell inoculation with human HCT116 colon adenocarcinoma cells followed by administration of saline (control; solid circles), free drug cocktail of CPT-11:FUDR at a 1:1 synergistic ratio (closed triangle) and a liposomal formulation of CPT-11:FUDR at a 1:1 synergistic ratio (open squares).

FIG. 8C is a graph of tumor weight versus time after tumor cell inoculation with human Capan-1 pancreatic tumor cells followed by administration of saline (control; solid circles), a free drug cocktail of CPT-11:FUDR at a 1:1 synergistic ratio (solid squares, inverted solid triangles), and a liposomal formulation of FUDR/CPT-11 at a 1:1 synergistic ratio (open squares).

### MODES OF CARRYING OUT THE INVENTION¹

The invention provides compositions comprising liposomes stably associated therewith at least one fluoropyrimidine and one water-soluble camptothecin, wherein the fluoropyrimidine and water-soluble camptothecin are present at fluoropyrimidine/camptothecin mole ratios that exhibit a desired cytotoxic, cytostatic or biologic effect to relevant cells or tumor cell homogenates. ¹ Abbreviations

DSPC: distearoylphosphatidylcholine; PG: phosphatidylglycerol; DSPG: distearoylphosphatidylglycerol; PI: phosphatidylinositol; Chol: cholesterol; CH or CHE: cholesteryl hexadecyl ether; DAPC: diarachidonoylphosphatidylcholineSUV:small unilamellar vesicle; LUV: large unilamellar vesicle; MLV: multilamellar vesicle; MTT: 3-(4,5-dimethylthiazol-2-yl)-2,5-diphenyl-2H tetrazolium bromide; EDTA: ethylenediaminetetraacetic acid; HEPES: N-[2-hydroxylethyl]-piperazine-N-[2-ethanesulfonic acid]; HBS: HEPES buffered saline (20 mM HEPES, 150 mM NaCl, pH 7.4); SHE: 300 mM sucrose, 20 mM HEPES, 30 mM EDTA; TEA: triethanolamine; CI: combination index; fₐ: fraction affected.

Preferably, liposomal compositions provided herein will include liposomes stably associated therewith at least one fluoropyrimidine and one water-soluble camptothecin in a mole ratio of the fluoropyrimidine/water-soluble camptothecin which exhibits a non-antagonistic effect to relevant cells or tumor cell homogenates.

Preferably, liposomal compositions of the invention will include liposomes stably associated therewith either 5-FU or FUDR and irinotecan. More preferably, 5-FU or FUDR and irinotecan will be present in compositions of the invention at a 5-FU (or FUDR):irinotecan mole ratio of between 100:1 and 1:100, even more preferably the mole ratio of 5-FU or FUDR to irinotecan will be in the range of 10:1 and 1:1.

In further embodiments of the invention, the above described lipid-based delivery vehicles comprise a third or fourth agent. Any therapeutic, diagnostic or cosmetic agent may be included.

In another aspect of the invention, liposomes which comprise a sterol are provided. Preferably the sterol is cholesterol.

The lipid-based delivery vehicles of the present invention may be used not only in parenteral administration but also in topical, nasal, subcutaneous, intraperitoneal, intramuscular, aerosol or oral delivery or by the application of the delivery vehicle onto or into a natural or synthetic implantable device at or near the target site for therapeutic purposes or medical imaging and the like. Preferably, the lipid-based delivery vehicles of the invention are used in parenteral administration, most preferably, intravenous administration. In another embodiment of the invention, leucovorin is administered with compositions of the invention in order to stabilize the fluoropyrimidines in vivo.

The preferred embodiments herein described are not intended to be exhaustive or to limit the scope of the invention to the precise forms disclosed. They are chosen and described to best explain the principles of the invention and its application and practical use to allow others skilled in the art to comprehend its teachings.

Water-Soluble Camptothecins

Camptothecins are a class of highly active anticancer drugs. The majority of these drugs are semi-synthetic or synthetic derivatives of the naturally occurring “Camptothecin” found in the bark of the Chinese camptotheca tree and the Asian nothapodytes tree. Camptothecins are reported to act by inhibiting the action of Topoisomerase I, an enzyme found in cells that is involved in the synthesis and replication of DNA. The enzyme is found in significantly higher amounts, and degrades more slowly, in many types of cancer cells as compared to normal cells. The clinical use of camptothecins has been limited due to an incomplete understanding of their mechanism of action and their poor water solubility. Nearly all naturally occurring camptothecins are poorly water-soluble; this property makes them difficult, and in many instances impossible, to formulate and administer. As a result, many camptothecins marketed or in development have been made water-soluble. It is generally accepted by those knowledgeable in the art that water-soluble camptothecins include those derivatives of Camptothecin that are charged at physiological pH. For example, enhanced water-solubility has been effectively achieved through addition of a hydrophilic hydroxyl or nitro group at the 9, 10, or 11 positions of the Camptothecin A ring. Similarly, addition of a positively charged dimethylaminomethyl group at the 9 position has demonstrated enhanced water-solubility.

Water-soluble derivatives of camptothecin have shown a broad spectrum of activity against human tumors. Because of this, the United States Food and Drug Administration (FDA) have approved water-soluble camptothecin formulations of irinotecan, topotecan and lurtotecan for clinical use in humans. The antitumor activity demonstrated with irinotecan (CPT-11) is thought to occur through its metabolite, SN-38 .

“Water-soluble camptothecins” of the invention refer to derivatives of Camptothecin or formulations thereof that are sufficiently soluble in water. Water-soluble camptothecins include, but are not limited to, irinotecan (CPT-11), SN-38, topotecan, 9-aminocamptothecin, lurtotecan and prodrugs, precursors, metabolic products of these drugs; as well as hydrophilic salt derivatives of water-insoluble camptothecins such as the sodium salt of the parent compound, Camptothecin. Preferably the water-soluble camptothecin for use in this invention is irinotecan, topotecan, 9-aminocamptothecin or lurtotecan. Most preferably, the water-soluble camptothecin is irinotecan.

Fluoropyrimidines

Fluoropyrimidine analogs of uracil, cytosine, or thymine, and the corresponding nucleosides are well known anticancer agents. Many such pyrimidine analogs or derivatives act as antimetabolites in that they closely resemble an essential metabolite and therefore interfere with physiological reactions involving it. A common mechanism of action for pyrimidine analogs is to inhibit the enzyme, thymidylate synthase. This inhibition prevents the methylation of dUMP (deoxyuridine monophosphate) and subsequent generation of dihydrofolate and thymidylate, which is an essential precursor in DNA synthesis. The result of this interference is inhibition of DNA biosynthesis. The role of thymidylate synthase in deoxynucleotide synthesis has made it a critical target enzyme in cancer chemotherapy, most specifically colorectal cancer and other neoplasms. Of particular interest is fluorinated pyrimidine analogs or “fluoropyrimidines”, such as fluorouracil (5-FU) and fluorodeoxyuridine (floxuridine or FUDR), which have been shown to have significant antitumor activity in humans.

“Fluoropyrimidines” as their name implies, refers to pyrimidine analogs that have been derivatized with a fluorine atom. Fluoropyrimidines of the present invention are recognized in the art as being equivalents of 5-FU or FUDR, including but not limited to, UFT (uracil-tegafur), Capecitabine, Futraful (FT-207) and prodrugs, precursors, metabolic products of 5-FU or FUDR such as FdUMP (5 fluoro-deoxyuridine monophosphate) and FUTP (fluoro-uridine triphosphate) and the like.

The combination of uracil and tegafur (UFT) has been used in clinical trials treating cancer for over two decades. Tegafur is metabolized in vivo into 5-FU while uracil interacts with the tegafur and prevents breakdown of 5-FU. Oral administration of UFT in some cases is an attractive alternative to 5-FU treatment as it is often associated with increased activity and reduced toxicity.

Capecitabine is a prodrug that is selectively tumor-activated to its cytotoxic moiety, fluorouracil, by thymidine phosphorylase. Fluorouracil is further metabolized to two active metabolites, 5-fluoro-2-deoxyuridine monophosphate (FdUMP) and 5-fluorouridine triphosphate (FUTP), within normal and tumor cells. FdUMP inhibits DNA synthesis by reducing normal thymidine production, while FUTP inhibits RNA and protein synthesis by competing with uridine triphosphate.

Preferably, fluoropyrimidines for use in the invention are 5-FU, FUDR or tegafur/uracil. More preferably, the fluoropyrimidine is FUDR or 5-FU. Most preferably, the fluoropyrimidine is FUDR. Leucovorin may be administered in conjunction with compositions of the invention. This compound has no antineoplastic activity; however, it is a standard practice of care with the FDA when treating patients with 5-FU as it results in a significant increase in the life span of 5-FU. Leucovorin acts by stabilizing the binding of 5-FU (and FUDR) to its target enzyme, thymidylate synthase, therefore protecting it from mechanisms that would otherwise lead to its clearance from the blood. The reduced clearance of the fluoropyrimidine allows it to exhibit a higher cytotoxic effect.

The effects of combining camptothecins and pyrimidine analogs in liposomes of the invention will act to at least inhibit both Topoisomerase I and thymidylate synthase, thus resulting in increased inhibition of DNA synthesis which is critical for the treatment of hyperproliferative diseases such as cancer.

Determining Non-Antagonistic Camptothecin/Fluoropyrimidine Ratios In Vitro

In a further aspect of the invention camptothecins and fluoropyrimidines will be encapsulated into liposomes at synergistic or additive (ie. non-antagonistic) ratios. Determination of ratios of agents that display synergistic or additive combination effects may be carried out using various algorithms, based on the types of experimental data described below. These methods include isobologram methods (Loewe, et al., Arzneim-Forsch (1953) 3:285-290; Steel, et al., Int. J. Radiol. Oncol. Biol. Phys. (1979) 5:27-55), the fractional product method (Webb, Enzyme and Metabolic Inhibitors (1963) Vol. 1, pp. 1-5. New York: Academic Press), the Monte Carlo simulation method, CombiTool, ComboStat and the Chou-Talalay median-effect method based on an equation described in Chou, J. Theor. Biol. (1976) 39:253-276; and Chou, Mol. Pharmacol. (1974) 10:235-247). Alternatives include surviving fraction (Zoli, et al., Int. J. Cancer (1999) 80:413-416), percentage response to granulocyte/macrophage-colony forming unit compared with controls (Pannacciulli, et al., Anticancer Res. (1999) 19:409-412) and others (Berenbaum, Pharmacol. Rev. (1989) 41:93-141; Greco, et al., Pharmacol Rev. (1995) 47:331-385).

The Chou-Talalay median-effect method is preferred. The analysis utilizes an equation wherein the dose that causes a particular effect, fₐ, is given by:

D=Dₘ[fₐ/(1−fₐ)]^{1/m}

in which D is the dose of the drug used, fₐ is the fraction of cells affected by that dose, Dₘ is the dose for median effect signifying the potency and m is a coefficient representing the shape of the dose-effect curve (m is 1 for first order reactions).

This equation can be further manipulated to calculate a combination index (CI) on the basis of the multiple drug effect equation as described by Chou and Talalay, Adv. Enzyme Reg. (1984) 22:27-55; and by Chou, et al., in: Synergism and Antagonism in Chemotherapy, Chou and Rideout, eds., Academic Press: New York 1991:223-244. A computer program (CalcuSyn) for this calculation is found in Chou and Chou (“Dose-effect analysis with microcomputers: quantitation of ED50, LD50, synergism, antagonism, low-dose risk, receptor ligand binding and enzyme kinetics”: CalcuSyn Manual and Software; Cambridge: Biosoft 1987).

The combination index equation is based on the multiple drug-effect equation of Chou-Talalay derived from enzyme kinetic models. An equation determines only the additive effect rather than synergism and antagonism. However, according to the CalcuSyn program, synergism is defined as a more than expected additive effect, and antagonism as a less than expected additive effect. Chou and Talalay in 1983 proposed the designation of CI=1 as the additive effect, thus from the multiple drug effect equation of two drugs, we obtain:

CI=(D)₁/(Dₓ)₁+(D)₂/(Dₓ)₂ [Eq. 1]

for mutually exclusive drugs that have the same or similar modes of action, and it is further proposed that

CI=(D)₁/(Dₓ)₁+(D)₂/(Dₓ)₂+((D)₁)(D)₂)/(Dₓ)₁(Dₓ)₂ [Eq. 2]

for mutually non-exclusive drugs that have totally independent modes of action. CI<1, =1, and >1 indicates synergism, additive effect, and antagonism, respectively. Equation 1 or equation 2 dictates that drug 1, (D)₁, and drug 2, (D)₂, (in the numerators) in combination inhibit x % in the actual experiment. Thus, the experimentally observed x % inhibition may not be a round number but most frequently has a decimal fraction. (Dₓ)₁ and (Dₓ)₂ (in the denominators) of equations 1 and 2 are the doses of drug 1 and drug 2 alone, respectively, inhibiting x %.

For simplicity, mutual exclusivity is usually assumed when more than two drugs are involved in combinations (CalcuSyn Manual and Software; Cambridge: Biosoft 1987).

A two-drug combination may be further used as a single pharmaceutical unit to determine synergistic or additive interactions with a third agent. In addition, a three-agent combination may be used as a unit to determine non-antagonistic interactions with a fourth agent, and so on.

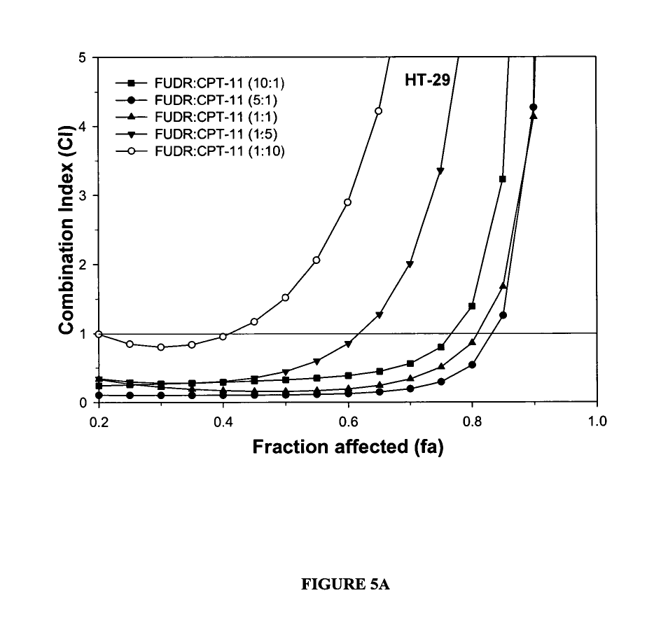

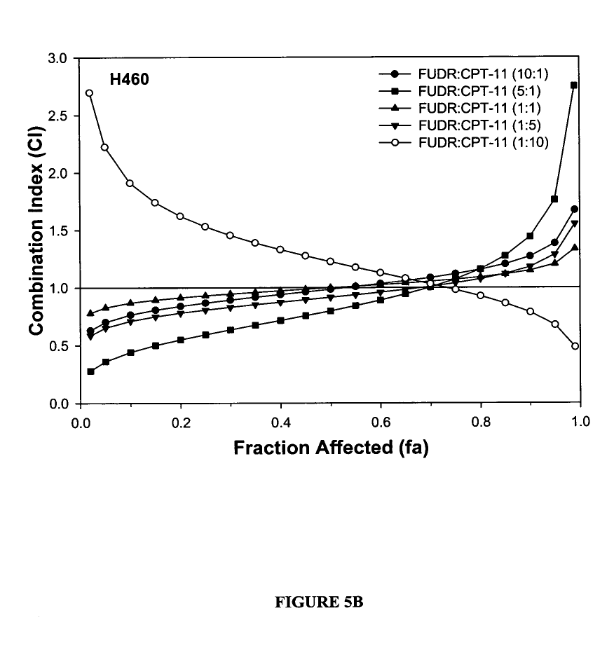

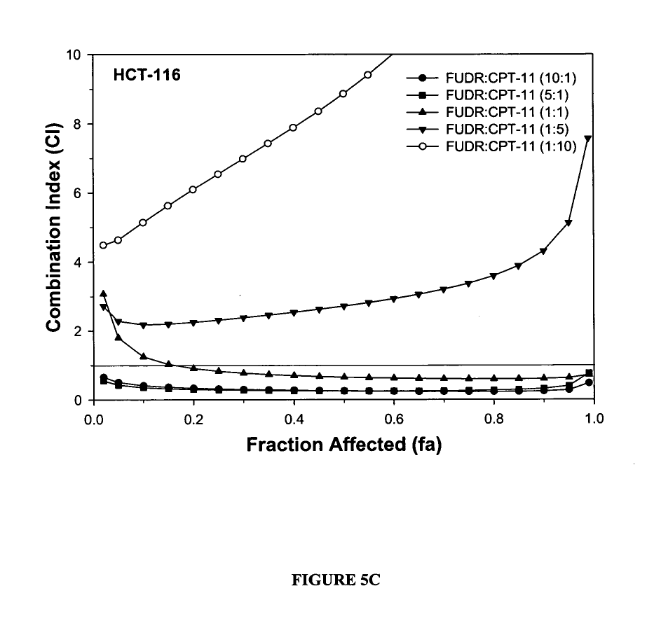

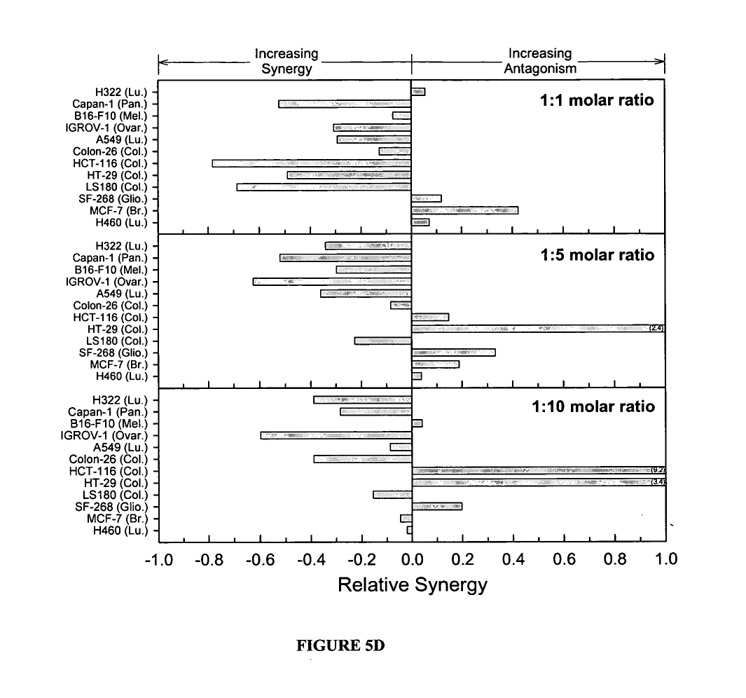

The underlying experimental data are generally determined in vitro using cells in culture or cell-free systems. Preferably, the combination index (CI) is plotted as a function of the fraction of cells affected (fₐ) as shown in FIG. 5A to 5C which, as explained above, is a surrogate parameter for concentration range. Preferred combinations of agents are those that display synergy or additivity over a substantial range of fₐ values. Combinations of agents are selected that display synergy over at least 5% of the concentration range wherein greater than 1% of the cells are affected, i.e., an fₐ range greater than 0.01. Preferably, a larger portion of overall concentration exhibits a favorable CI; for example, 5% of an fₐ range of 0.2-1.0. More preferably 10% of this range exhibits a favorable CI. Even more preferably, 20% of the fₐ range, preferably over 50% and most preferably over at least 70% of the fₐ range of 0.2 to 1.0 are utilized in the compositions. Combinations that display synergy over a substantial range of fₐ values may be re-evaluated at a variety of agent ratios to define the optimal ratio to enhance the strength of the non-antagonistic interaction and increase the fₐ range over which synergy is observed.

While it would be desirable to have synergy over the entire range of concentrations over which cells are affected, it has been observed that in many instances, the results are considerably more reliable in an fₐ range of 0.2-0.8. Thus, although the synergy exhibited by combinations of the invention is set forth to exist within the broad range of 0.01 or greater, it is preferable that the synergy be established in the fₐ range of 0.2-0.8. Other more sensitive assays, however, can be used to evaluate synergy at fₐ values greater than 0.8 for example bioluminescence or clonogenecity assays.

The optimal combination ratio may be further used as a single pharmaceutical unit to determine synergistic or additive interactions with a third agent. In addition, a three-agent combination may be used as a unit to determine non-antagonistic interactions with a fourth agent, and so on.

As set forth above, the in vitro studies on cell cultures will be conducted with “relevant” cells. The choice of cells will depend on the intended therapeutic use of the agent. Only one relevant cell line or cell culture type need exhibit the required non-antagonistic effect in order to provide a basis for the compositions to come within the scope of the invention.

For example, in one preferred embodiment of the invention, the combination of agents is intended for anticancer therapy. Appropriate choices will then be made of the cells to be tested and the nature of the test. In particular, tumor cell lines are suitable subjects and measurement of cell death or cell stasis is an appropriate end point. As will further be discussed below, in the context of attempting to find suitable non-antagonistic combinations for other indications, other target cells and criteria other than cytotoxicity or cell stasis could be employed.

For determinations involving antitumor agents, cell lines may be obtained from standard cell line repositories (NCI or ATCC for example), from academic institutions or other organizations including commercial sources. Preferred cell lines would include one or more selected from cell lines identified by the Developmental Therapeutics Program of the NCI/NIH. The tumor cell line screen used by this program currently identifies 60 different tumor cell lines representing leukemia, melanoma, and cancers of the lung, colon, brain, ovary, breast, prostate and kidney. The required non-antagonistic effect over a desired concentration range need be shown only on a single cell type; however, it is preferred that at least two cell lines exhibit this effect, more preferably three cell lines, more preferably five cell lines, and more preferably 10 cell lines. The cell lines may be established tumor cell lines or primary cultures obtained from patient samples. The cell lines may be from any species but the preferred source will be mammalian and in particular human. The cell lines may be genetically altered by selection under various laboratory conditions, and/or by the addition or deletion of exogenous genetic material. Cell lines may be transfected by any gene-transfer technique, including but not limited to, viral or plasmid-based transfection methods. The modifications may include the transfer of cDNA encoding the expression of a specific protein or peptide, a regulatory element such as a promoter or enhancer sequence or antisense DNA or RNA. Genetically engineered tissue culture cell lines may include lines with and without tumor suppressor genes, that is, genes such as p53, pTEN and p16; and lines created through the use of dominant negative methods, gene insertion methods and other selection methods. Preferred tissue culture cell lines that may be used to quantify cell viability, e.g., to test antitumor agents, include, but are not limited to, H460, MCF-7, SF-268, HT29, HCT-116, LS180, B16-F10, A549, Capan pancreatic, CAOV-3, IGROV1, PC-3, MX-1 and MDA-MB-231.

In one preferred embodiment, the given effect (fₐ) refers to cell death or cell stasis after application of a cytotoxic agent to a cell culture. Cell death or viability may be measured, for example, using the following methods:

| CYTOTOXICITY ASSAY | REFERENCE |
|---|---|
| MTT assay | Mosmann, J. Immunol. Methods |
| | (1983) 65(1-2): 55-63. |
| Trypan blue dye exclusion | Bhuyan, et al., Experimental Cell Research |
| | (1976) 97: 275-280. |
| Radioactive tritium (³H)-thymidine | Senik, et al., Int. J. Cancer |
| incorporation or DNA intercalating assay | (1975) 16(6): 946-959. |
| Radioactive chromium-51 release assay | Brunner, et al., Immunology |
| | (1968) 14: 181-196. |
| Glutamate pyruvate transaminase, creatine | Mitchell, et al., J. of Tissue Culture Methods |
| phosphokinase and lactate dehydrogenase | (1980) 6(3&4): 113-116. |
| enzyme leakage | |
| Neutral red uptake | Borenfreund and Puerner, Toxicol. Lett. |
| | (1985) 39: 119-124. |
| Alkaline phosphatase activity | Kyle, et al., J. Toxicol. Environ. Health |
| | (1983) 12: 99-117. |
| Propidium iodide staining | Nieminen, et al., Toxicol. Appl. Pharmacol. |
| | (1992) 115: 147-155. |
| Bis-carboxyethyl-carboxyfluorescein (BCECF) | Kolber, et al., J. Immunol. Methods |
| retention | (1988) 108: 255-264. |
| Mitochondrial membrane potential | Johnson, et al., Proc. Natl. Acad. Sci. USA |
| | (1980) 77: 990-994. |
| Clonogenic Assays | Puck, et al., J. of Experimental Medicine |
| | (1956) 103: 273-283. |
| LIVE/DEAD Viability/Cytotoxicity assay | Morris, Biotechniques (1990) 8: 296-308. |
| Sulforhodamine B (SRB) assays | Rubinstein, et al., J. Natl. Cancer Instit. |
| | (1990) 82: 1113-1118. |

The “MTT assay” is preferred.

Non-antagonistic ratios of two or more agents can be determined for disease indications other than cancer and this information can be used to prepare therapeutic formulations of two or more drugs for the treatment of these diseases. With respect to in vitro assays, many measurable endpoints can be selected from which to define drug synergy, provided those endpoints are therapeutically relevant for the specific disease.

As set forth above, the in vitro studies on cell cultures will be conducted with “relevant” cells. The choice of cells will depend on the intended therapeutic use of the agent. In vitro studies on individual patient biopsies or whole tumors will be conducted with “tumor cell homogenates,” generated from the mechanical or chemical disruption of the tumor sample(s) into single, whole cells.

In one preferred embodiment, the given effect (fₐ) refers to cell death or cell stasis after application of a cytotoxic agent to a “relevant” cell culture or “tumor cell homogenate” (see Example 4). Cell death or viability may be measured using a number of methods known in the art. The “MTT” assay (Mosmann, J. Immunol. Methods (1983) 65(1-2):55-63) detailed in Example 4 is preferred.

Preparation of Lipid-Based Delivery Vehicles

Preferred lipid carriers for use in this invention are liposomes. Liposomes can be prepared as described in Liposomes: Rational Design (A. S. Janoff, ed., Marcel Dekker, Inc., New York, N.Y.), or by additional techniques known to those knowledgeable in the art. Suitable liposomes for use in this invention include large unilamellar vesicles (LUVs), multilamellar vesicles (MLVs), small unilamellar vesicles (SUVs) and interdigitating fusion liposomes.

Liposomes for use in this invention may be prepared to be of “low-cholesterol.” Such liposomes are “cholesterol free,” or contain “substantially no cholesterol,” or “essentially no cholesterol.” The term “cholesterol free” as used herein with reference to a liposome means that a liposome is prepared in the absence of cholesterol. The term “substantially no cholesterol” allows for the presence of an amount of cholesterol that is insufficient to significantly alter the phase transition characteristics of the liposome (typically less than 20 mol % cholesterol). The incorporation of less than 20 mol % cholesterol in liposomes can allow for retention of drugs not optimally retained when liposomes are prepared with greater than 20 mol % cholesterol. Preferably, liposomes of the invention contain some cholesterol. Additionally, liposomes prepared with less than 20 mol % cholesterol display narrow phase transition temperatures, a property that may be exploited for the preparation of liposomes that release encapsulated agents due to the application of heat (thermosensitive liposomes). Liposomes of the invention may also contain therapeutic lipids, which examples include ether lipids, phosphatidic acid, phosphonates, ceramide and ceramide analogs, sphingosine and sphingosine analogs and serine-containing lipids.

Liposomes may also be prepared with surface stabilizing hydrophilic polymer-lipid conjugates such as polyethylene glycol-DSPE, to enhance circulation longevity. The incorporation of negatively charged lipids such as phosphatidylglycerol (PG) and phosphatidylinositol (PI) may also be added to liposome formulations to increase the circulation longevity of the carrier. These lipids may be employed to replace hydrophilic polymer-lipid conjugates as surface stabilizing agents. Preferred embodiments of this invention may make use of low-cholesterol liposomes containing PG or PI to prevent aggregation thereby increasing the blood residence time of the carrier.

In one embodiment, liposome compositions in accordance with this invention are preferably used to treat cancer, including multi-drug resistant cancers such as 5-FU resistant colorectal cancer. Delivery of encapsulated drugs to a tumor site is achieved by administration of liposomes of the invention. Preferably liposomes have a diameter of less than 300 nm. Most preferably liposomes have a diameter of less than 200 nm. Tumor vasculature is generally leakier than normal vasculature due to fenestrations or gaps in the endothelia. This allows the delivery vehicles of 200 nm or less in diameter to penetrate the discontinuous endothelial cell layer and underlying basement membrane surrounding the vessels supplying blood to a tumor. Selective accumulation of the delivery vehicles into tumor sites following extravasation leads to enhanced anticancer drug delivery and therapeutic effectiveness.

Various methods may be utilized to encapsulate active agents in liposomes. “Encapsulation,” includes covalent or non-covalent association of an agent with the lipid-based delivery vehicle. For example, this can be by interaction of the agent with the outer layer or layers of the liposome or entrapment of an agent within the liposome, equilibrium being achieved between different portions of the liposome. Thus encapsulation of an agent can be by association of the agent by interaction with the bilayer of the liposomes through covalent or non-covalent interaction with the lipid components or entrapment in the aqueous interior of the liposome, or in equilibrium between the internal aqueous phase and the bilayer. “Loading” refers to the act of encapsulating one or more agents into a delivery vehicle.

Encapsulation of the desired combination can be achieved either through encapsulation in separate delivery vehicles or within the same delivery vehicle. Where encapsulation into separate liposomes is desired, the lipid composition of each liposome may be quite different to allow for coordinated pharmacokinetics. By altering the vehicle composition, release rates of encapsulated drugs can be matched to allow desired ratios of the drugs to be delivered to the tumor site. Means of altering release rates include increasing the acyl-chain length of vesicle forming lipids to improve drug retention, controlling the exchange of surface grafted hydrophilic polymers such as PEG out of the liposome membrane and incorporating membrane-rigidifying agents such as sterols or sphingomyelin into the membrane. It should be apparent to those skilled in the art that if a first and second drug are desired to be administered at a specific drug ratio and if the second drug is retained poorly within the liposome composition of the first drug (e.g., DMPC/Chol), that improved pharmacokinetics may be achieved by encapsulating the second drug in a liposome composition with lipids of increased acyl chain length (e.g., DSPC/Chol). When encapsulated in separate liposomes, it should be readily accepted that ratios of water-soluble camptothecins-to-fluoropyrimidines that have been determined on a patient-specific basis to provide optimal therapeutic activity, would be generated for individual patients by combining the appropriate amounts of each liposome-encapsulated drug prior to administration. Alternatively, two or more agents may be encapsulated within the same liposome.

Techniques for encapsulation are dependent on the nature of the delivery vehicles. For example, therapeutic agents may be loaded into liposomes using both passive and active loading methods. Passive methods of encapsulating active agents in liposomes involve encapsulating the agent during the preparation of the liposomes. This includes a passive entrapment method described by Bangham, et al. (J. Mol. Biol. (1965) 12:238). This technique results in the formation of multilamellar vesicles (MLVs) that can be converted to large unilamellar vesicles (LUVs) or small unilamellar vesicles (SUVs) upon extrusion. Additional suitable methods of passive encapsulation include an ether injection technique described by Deamer and Bangham (Biochim. Biophys. Acta (1976) 443:629) and the Reverse Phase Evaporation technique as described by Szoka and Paphadjopoulos (P.N.A.S. (1978) 75:4194).

Active methods of encapsulation include the pH gradient loading technique described in U.S. Pat. Nos. 5,616,341, 5,736,155 and 5,785,987 and active metal-loading. A preferred method of pH gradient loading is the citrate-base loading method utilizing citrate as the internal buffer at a pH of 4.0 and a neutral exterior buffer. Other methods employed to establish and maintain a pH gradient across a liposome involve the use of an ionophore that can insert into the liposome membrane and transport ions across membranes in exchange for protons (see U.S. Pat. No. 5,837,282). A recent technique utilizing transition metals to drive the uptake of drugs into liposomes via complexation in the absence of an ionophore may also be used. This technique relies on the formation of a drug-metal complex rather than the establishment of a pH gradient to drive uptake of drug.

Metal-based active loading typically uses liposomes with passively encapsulated metal ions (with or without passively loaded therapeutic agents). Various salts of metal ions are used, presuming that the salt is pharmaceutically acceptable and soluble in an aqueous solutions. Actively loaded agents are selected based on being capable of forming a complex with a metal ion and thus being retained when so complexed within the liposome, yet capable of loading into a liposome when not complexed to metal ions. Agents that are capable of coordinating with a metal typically comprise coordination sites such as amines, carbonyl groups, ethers, ketones, acyl groups, acetylenes, olefins, thiols, hydroxyl or halide groups or other suitable groups capable of donating electrons to the metal ion thereby forming a complex with the metal ion. Examples of active agents which bind metals include, but are not limited to, quinolones such as fluoroquinolones; quinolones such as nalidixic acid; anthracyclines such as doxorubicin, daunorubicin and idarubicin; amino glycosides such as kanamycin; and other antibiotics such as bleomycin, mitomycin C and tetracycline; and nitrogen mustards such as cyclophosphamide, thiosemicarbazones, indomethacin and nitroprusside; camptothecins such as topotecan, irinotecan, lurtotecan, 9-aminocamptothecin, 9-nitrocamptothecin and 10-hydroxycamptothecin; and podophyllotoxins such as etoposide. Uptake of an agent may be established by incubation of the mixture at a suitable temperature after addition of the agent to the external medium. Depending on the composition of the liposome, temperature and pH of the internal medium, and chemical nature of the agent, uptake of the agent may occur over a time period of minutes or hours. Methods of determining whether coordination occurs between an agent and a metal within a liposome include spectrophotometric analysis and other conventional techniques well known to those of skill in the art. Preferably, liposomes of the invention will contain a metal ion solution. Preferably the metal ion will be copper.

Passive and active methods of entrapment may also be coupled in order to prepare a liposome formulation containing more than one encapsulated agent.

Administering Compositions of the Invention In Vivo

As mentioned above, the delivery vehicle compositions of the present invention may be administered to warm-blooded animals, including humans as well as to domestic avian species. For treatment of human ailments, a qualified physician will determine how the compositions of the present invention should be utilized with respect to dose, schedule and route of administration using established protocols. Such applications may also utilize dose escalation should agents encapsulated in delivery vehicle compositions of the present invention exhibit reduced toxicity to healthy tissues of the subject.

Preferably, the pharmaceutical compositions of the present invention are administered parenterally, i.e., intraarterially, intravenously, intraperitoneally, subcutaneously, or intramuscularly. More preferably, the pharmaceutical compositions are administered intravenously or intraperitoneally by a bolus injection. For example, see Rahman, et al., U.S. Pat. No. 3,993,754; Sears, U.S. Pat. No. 4,145,410; Papahadjopoulos, et al., U.S. Pat. No. 4,235,871; Schneider, U.S. Pat. No. 4,224,179; Lenk, et al., U.S. Pat. No. 4,522,803; and Fountain, et al., U.S. Pat. No. 4,588,578, incorporated by reference.

In other methods, the pharmaceutical or cosmetic preparations of the present invention can be contacted with the target tissue by direct application of the preparation to the tissue. The application may be made by topical, “open” or “closed” procedures. By “topical” , it is meant the direct application of the multi-drug preparation to a tissue exposed to the environment, such as the skin, oropharynx, external auditory canal, and the like. “Open” procedures are those procedures that include incising the skin of a patient and directly visualizing the underlying tissue to which the pharmaceutical preparations are applied. This is generally accomplished by a surgical procedure, such as a thoracotomy to access the lungs, abdominal laparotomy to access abdominal viscera, or other direct surgical approach to the target tissue. “Closed” procedures are invasive procedures in which the internal target tissues are not directly visualized, but accessed via inserting instruments through small wounds in the skin. For example, the preparations may be administered to the peritoneum by needle lavage. Alternatively, the preparations may be administered through endoscopic devices.

Pharmaceutical compositions comprising delivery vehicles of the invention are prepared according to standard techniques and may comprise water, buffered water, 0.9% saline, 0.3% glycine, 5% dextrose and the like, including glycoproteins for enhanced stability, such as albumin, lipoprotein, globulin, and the like. These compositions may be sterilized by conventional, well-known sterilization techniques. The resulting aqueous solutions may be packaged for use or filtered under aseptic conditions and lyophilized, the lyophilized preparation being combined with a sterile aqueous solution prior to administration. The compositions may contain pharmaceutically acceptable auxiliary substances as required to approximate physiological conditions, such as pH adjusting and buffering agents, tonicity adjusting agents and the like, for example, sodium acetate, sodium lactate, sodium chloride, potassium chloride, calcium chloride, and the like. Additionally, the delivery vehicle suspension may include lipid-protective agents which protect lipids against free-radical and lipid-peroxidative damages on storage. Lipophilic free-radical quenchers, such as alpha-tocopherol and water-soluble iron-specific chelators, such as ferrioxamine, are suitable. Leucovorin may also be administered with compositions of the invention through standard techniques to enhance the life span of administered fluoropyrimidines.

The concentration of delivery vehicles in the pharmaceutical formulations can vary widely, such as from less than about 0.05%, usually at or at least about 2-5% to as much as 10 to 30% by weight and will be selected primarily by fluid volumes, viscosities, and the like, in accordance with the particular mode of administration selected. For example, the concentration may be increased to lower the fluid load associated with treatment. Alternatively, delivery vehicles composed of irritating lipids may be diluted to low concentrations to lessen inflammation at the site of administration. For diagnosis, the amount of delivery vehicles administered will depend upon the particular label used, the disease state being diagnosed and the judgment of the clinician.

Preferably, the pharmaceutical compositions of the present invention are administered intravenously. Dosage for the delivery vehicle formulations will depend on the ratio of drug to lipid and the administrating physician's opinion based on age, weight, and condition of the patient.

In addition to pharmaceutical compositions, suitable formulations for veterinary use may be prepared and administered in a manner suitable to the subject. Preferred veterinary subjects include mammalian species, for example, non-human primates, dogs, cats, cattle, horses, sheep, and domesticated fowl. Subjects may also include laboratory animals, for example, in particular, rats, rabbits, mice, and guinea pigs.

Kits

The therapeutic agents in the invention compositions may be formulated separately in individual compositions wherein each therapeutic agent is stably associated with appropriate delivery vehicles. These compositions can be administered separately to subjects as long as the pharmacokinetics of the delivery vehicles are coordinated so that the ratio of therapeutic agents administered is maintained at the target for treatment. Thus, it is useful to construct kits which include, in separate containers, a first composition comprising delivery vehicles stably associated with at least a first therapeutic agent and, in a second container, a second composition comprising delivery vehicles stably associated with at least one second therapeutic agent. The containers can then be packaged into the kit.

The kit will also include instructions as to the mode of administration of the compositions to a subject, at least including a description of the ratio of amounts of each composition to be administered. Alternatively, or in addition, the kit is constructed so that the amounts of compositions in each container is pre-measured so that the contents of one container in combination with the contents of the other represent the correct ratio. Alternatively, or in addition, the containers may be marked with a measuring scale permitting dispensation of appropriate amounts according to the scales visible. The containers may themselves be useable in administration; for example, the kit might contain the appropriate amounts of each composition in separate syringes. Formulations which comprise the pre-formulated correct ratio of therapeutic agents may also be packaged in this way so that the formulation is administered directly from a syringe prepackaged in the kit.

The following examples are offered to illustrate but not to limit the invention.

### EXAMPLES

Methods for Preparation of Large Unilamellar Liposomes

Unless otherwise specified, lipids were dissolved in chloroform solution and subsequently dried under a stream of nitrogen gas and placed in a vacuum pump to remove solvent. Trace levels of radioactive lipid ¹⁴C-CHE were added to quantify lipid. The resulting lipid film was placed under high vacuum for a minimum of 2 hours. The lipid film was hydrated in the solution indicated to form multilamellar vesicles (MLVs). The resulting preparation was extruded 10 times through stacked polycarbonate filters with an extrusion apparatus (Lipex Biomembranes, Vancouver, BC) to achieve a mean liposome size between 80 and 150 nm. All constituent lipids of liposomes are reported in mole %.

Methods for Quantification of Drug Loading

At various time points after initiation of drug loading, aliquots were removed and passed through a Sephadex G-50 spin column to separate free from encapsulated drug. To a specified volume of eluant, Triton X-100 or N-ocyl beta-D-glucopyranoside (OGP) was added to solubilize the liposomes. Following addition of detergent, the mixture was heated to the cloud point of the detergent and allowed to cool to room temperature before measurement of the absorbance or fluorescence. Drug concentrations were calculated by comparison to a standard curve. Lipid levels were measured by liquid scintillation counting.

### Example 1A

### CPT-11 and FUDR can be Dual-Loaded into Liposomes (Lipid Film Method)

In order to determine whether workable liposomes containing both a water-soluble camptothecin and a fluoropyrimidine could be generated, DSPC/DSPG/Chol (70:20:10 mole ratio) liposomes containing passively entrapped FUDR were actively loaded with CPT-11.

Lipid films were prepared by dissolving DSPC to 50 mg/ml, cholesterol to 50 mg/ml in chloroform, and DSPG to 25 mg/ml in chloroform/methanol/water (50/10/1). The lipids were then combined and following solvent removal the resulting lipid films were hydrated with a solution consisting of 100 mM Cu(gluconate)₂, 220 mM triethanolamine (TEA), pH 7.4 and 30 mg/mL (122 mM) of FUDR (with trace amounts of ³H-FUDR) at 70° C. The resulting MLVs were extruded at 70° C. to generate LUVs. The mean diameter of the resulting liposomes was determined by QELS (quasi-elastic light scattering) analysis to be approximately 100 nm+/−20 nm. Subsequently, the liposomes were buffer exchanged into 300 mM sucrose, 20 mM Hepes, 30 mM EDTA (SHE), pH 7.4, using a hand-held tangential flow column and then into saline, thus removing any unencapsulated FUDR and Cu(gluconate)₂.

CPT-11 was added to these liposomes such that the FUDR to CPT-11 mole ratio would be 1:1. Loading of CPT-11 into the liposomes with an initial CPT-11 to lipid ratio of 0.1:1 was facilitated by incubating the samples at 50° C. for 10 minutes. After loading and then cooling to room temperature, the samples were exchanged into saline (0.9% Sodium Chloride Injection, USP; pH 5.5, Baxter), by tangential flow dialysis to remove EDTA or unencapsulated drug(s). The extent of CPT-11 loading was measured using absorbance at 370 nm against a standard curve. A drug to lipid ratio at each time point was generated using liquid scintillation counting to determine lipid concentrations (¹⁴C-CHE) and FUDR concentrations (³H-FUDR).

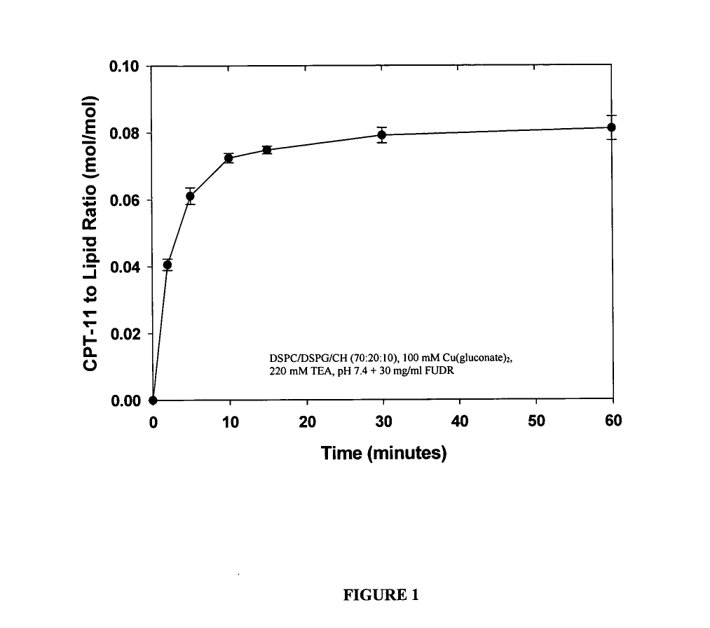

FIG. 1 shows the mean drug/lipid ratio (+/−standard deviation) over time during loading of CPT-11 at 50° C. It is apparent from FIG. 1 that CPT-11, added at an initial CPT-11 to lipid ratio of 0.1:1, can be actively loaded into DSPC/DSPG/Chol (70:20:10 mole ratio) liposomes, containing passively entrapped FUDR, to a final CPT-11 to lipid ratio of about 0.8:1. These results thus demonstrate that DSPC/DSPG/Chol (70:20:10 mole ratio) liposomes with encapsulated FUDR can be effectively dual-loaded with CPT-11.

### Example 1B

### CPT-11 and FUDR Loaded into Liposomes Prepared by Organic Emulsion Method

The required amounts of the lipids DSPC/DSPG/Cholesterol (70:20:10) are weighed out and dissolved in a Dichloromethane/Methanol/Water mixture (93/5/2) to a final concentration of 125 mg/mL. The dissolved lipids are placed into a glass Applikon bioreactor and nitrogen is gently bubbled through the mixture. A 100 mM Copper Gluconate/180 mM TEA buffer is then pumped into the Applikon bioreactor in order to provide a final lipid concentration of 75 mg/mL. While the Copper buffer is being pumped into the bioreactor, the entire mixture is agitated with heavy vortex mixing for a minimum of 90 minutes with continual nitrogen sparging. The temperature of the bioreactor is maintained at 70 degrees Celsius for the entire process by an external water bath hooked up to the bioreactor.

Liposome formation is followed by temperature and visual inspection of the mixture looking for, in sequence, water-in-oil emulsion, a “pudding” phase, “breaking” of the pudding phase, and establishment of a homogeneous liposome suspension. The crude liposomes are then extruded through 0.1 μm pore size filters under pressure at 70° C. until the mean liposome size is less than 150 nm and preferably between 110 and 125 nm with 90%<200 nm (analysis by dynamic light scattering). Filtration to remove external copper is completed against 10 volumes of sucrose phosphate EDTA buffer at room temperature using tangential flow hollow fiber filters.

Co-loading of Floxuridine and Irinotecan HCl Trihydrate requires dissolving the Floxuridine in sucrose phosphate EDTA buffer at pH 7.0 and then adding the Irinotecan HCl Trihydrate to the dissolved Floxuridine with vigorous mixing and heating to 50° C. if required to dissolve the Irinotecan HCl Trihydrate. The dissolved drugs in buffer are added to the liposomes at the required concentrations in a glass vessel. Loading proceeds for 1 hour at 50° C. with continuous mixing of the system. The liposome-drug mixture is allowed to cool to room temperature before proceeding with filtration. Filtration to remove unencapsulated drug is completed against 10 volumes of sucrose phosphate buffer at room temperature using tangential flow hollow fiber filters.

The liposomes with encapsulated drugs are diluted with sucrose phosphate buffer to 5 mg/mL Irinotecan HCl Trihydrate at a 1:1 mol ratio with Floxuridine. The dilution is followed by sterile filtration through 0.2 μm filters.

### Example 2

### Dual-Loaded Liposomes can be Prepared Using Intraliposomal Solutions of Varying Compositions

To examine the effects of internal liposomal solution on dual loading, DSPC/DSPG/Chol (70:20:10 mole ratio) liposomes containing various copper solutions were prepared. FUDR was passively entrapped and the extent of CPT-11 loading was measured.

Lipid films were prepared as described above except that lipid films were hydrated in 1 mL of either 100 mM copper gluconate, 220 mM TEA, pH 7.4; 100 mM CuSO₄, 265 mM TEA, pH 7.4; or 150 mM copper tartrate, 20 mM Hepes, pH 7.4 containing approximately 25 mg/ml FUDR (with trace amounts of ³H-FUDR). The resulting MLVs were extruded at 70° C. The mean diameter of each sample was determined by QELS (quasi-elastic light scattering) analysis to be approximately 100 nm+/−20 nm. The liposomes were then buffer exchanged into SHE, pH 7.4 using a hand-held tangential flow column and then into HBS, pH 7.4.

The CPT-11 uptake experiment was performed as previously described. CPT-11 was added to liposomes at an initial CPT-11 to lipid mole ratio of 0.1:1. Loading was facilitated by pre-heating the samples at 50° C. for 1 minute. A drug to lipid ratio for each time point was generated using liquid scintillation counting to determine lipid concentrations (¹⁴C-CHE) and FUDR concentrations (³H-FUDR). Absorbance at 370 nm against a standard curve was used to determine CPT-11 concentrations.

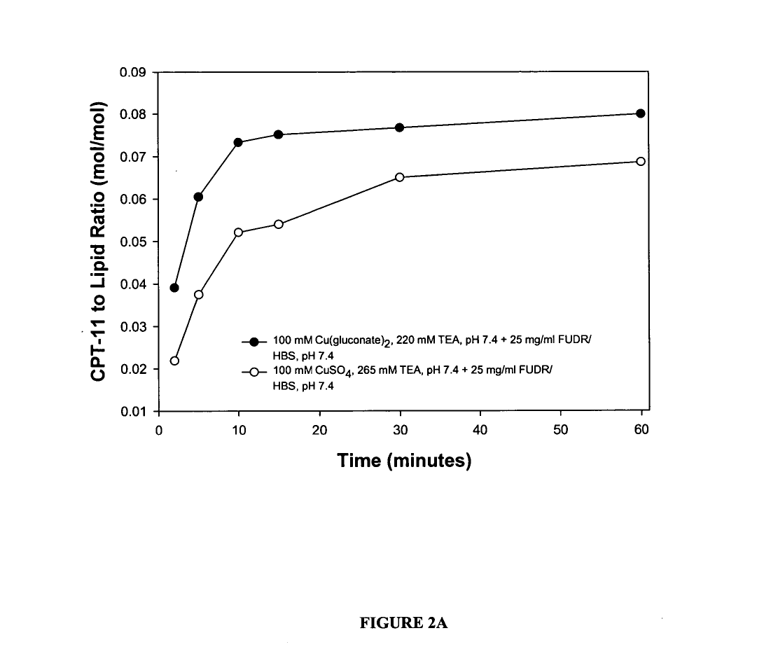

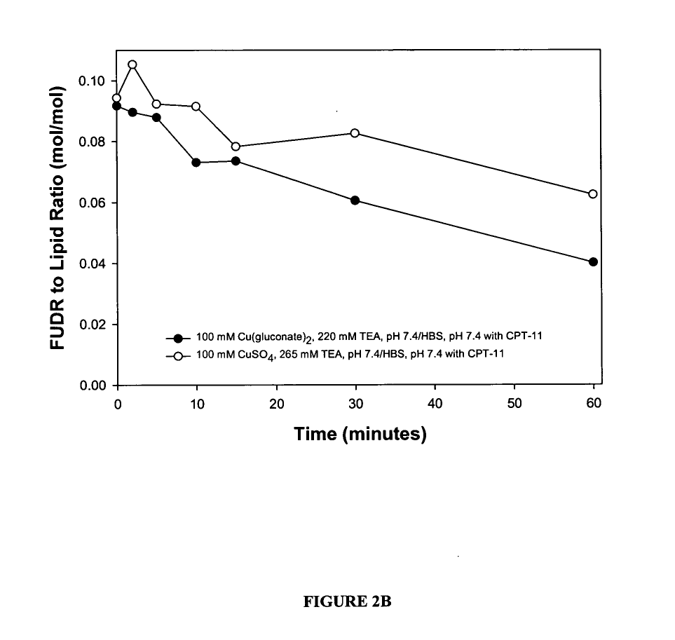

FIG. 2A shows that DSPC/DSPG/Chol (70:20:10 mole ratio) liposomes, containing passively entrapped FUDR, exhibit sufficient loading of CPT-11 with intraliposomal solutions of either copper gluconate or copper sulphate. However, copper gluconate appears to be a slightly more efficient solution for actively loading CPT-11 into liposomes of this nature. Similarly, release of FUDR over time from these dual-loaded liposomes is comparable in liposomes prepared with either copper gluconate or copper sulphate (FIG. 2B) as both show a gradual release of drug.

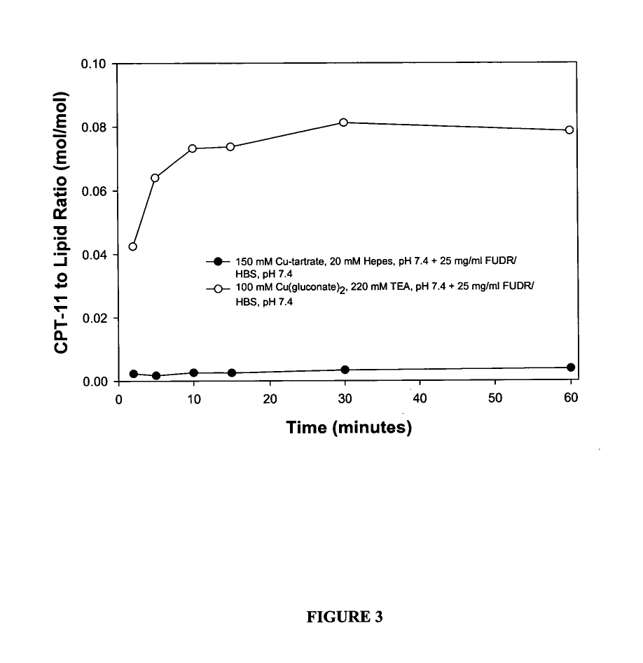

As demonstrated in FIG. 3, copper tartrate alone is not a suitable intraliposomal solution for dual loading CPT-11 into FUDR-containing liposomes. It is readily apparent from the graph in FIG. 3 that CPT-11 is unable to load into DSPC/DSPG/Chol liposomes containing passively entrapped FUDR and 150 mM copper tartrate.

### Example 3

### Presence of a Cholesterol Affects Plasma Concentrations of CPT-11 and FUDR

It is well known in the art that cholesterol and other sterols are routinely used in delivery vehicle preparations, such as liposomes, in order to broaden the range of temperatures at which phase transition occurs, decrease lipid aggregation and possibly alter circulation lifetimes of delivery vehicles. Generally, it is believed that greater than 30 mol % cholesterol is required to achieve the benefits of this sterol in liposomal preparations. To study the effects of cholesterol content on drug retention and pharmacokinetics of dual-loaded liposomes, DSPC/DSPG/Chol liposomes were prepared using varying amounts of cholesterol. A fluoropyrimidine was first passively entrapped into each set of liposomes, followed by active loading of a water-soluble camptothecin. The drug to lipid ratio for each drug was determined in the plasma of Balb/c mice in order to measure the extent of liposomal drug retention in vivo for each formulation over time.

Lipid films were prepared by dissolving DSPC and cholesterol in chloroform and DSPG in chloroform/methanol/water (16/8/1). The lipids were combined at mole ratios of DSPC/Chol/DSPG (75:5:20), DSPC/Chol/DSPG (70:10:20), DSPC/Chol/DSPG (65:15:20), and DSPC/Chol/DSPG (60:20:20) with trace amounts of ¹⁴C-CHE added as a liposomal lipid marker. After solvent removal, lipid films were hydrated in 250 mM CuSO₄ containing 25 mg/ml FUDR (with trace amounts of ³H-FUDR). The resulting MLVs were extruded at 70° C. The mean diameter of each sample was determined by QELS (quasi-elastic light scattering) analysis to be approximately 100 nm+/−20 nm. The liposomes were then buffer exchanged into SHE, pH 7.4 using a hand-held tangential flow column.

Liposomes were loaded similar to before with CPT-11 at 50° C. for 5 minutes. CPT-11 , FUDR and lipid levels were determined as described in Example 1. After loading of CPT-11 and cooling to room temperature, aliquotted samples of 340 μmol lipid/kg, 34 μmol FUDR/kg, and 34 μmol CTP-11/kg were injected into the tail vein of female Balb/c mice. At 1, 4, and 24 hours after intravenous administration, blood was collected by cardiac puncture and placed into EDTA coated microcontainers. Three mice were used for each time point. The samples were centrifuged to separate plasma and plasma was then carefully transferred to another tube. Plasma lipid and FUDR concentrations were determined by liquid scintillation counting, and plasma CPT-11 concentrations were determined by High Performance Liquid Chromatography (HPLC).

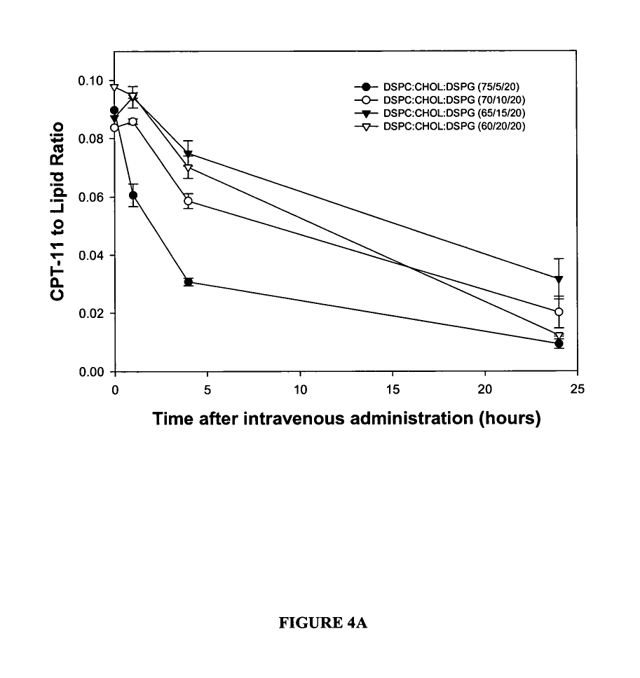

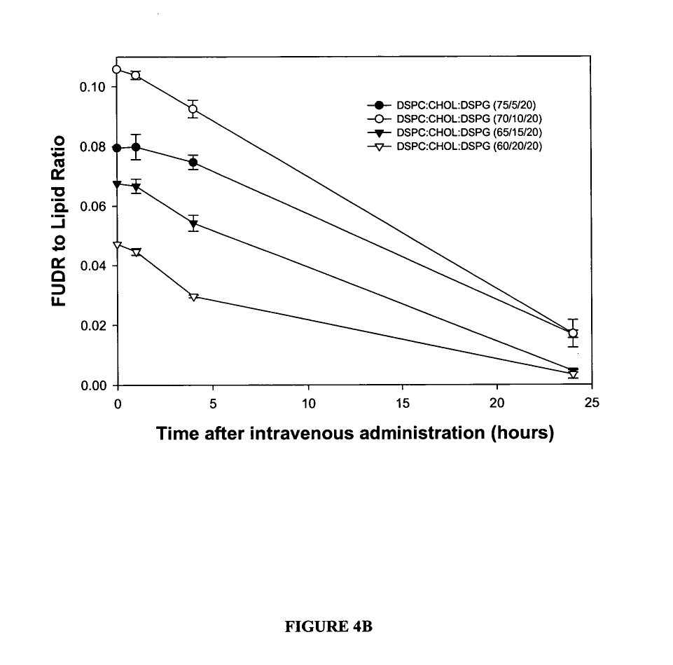

FIG. 4A illustrates the CPT-11 to lipid ratio (+/−standard deviation) as a function of time after intravenous administration of liposome-encapsulated CPT-11 and FUDR to Balb/c mice. The graph demonstrates that as the concentration of liposomal cholesterol is increased, there is a corresponding increase in in vivo liposome retention of CPT-11, with the exception of 20 mole % cholesterol which, compared to 15 mole %, had reduced CPT-11 levels and faster release. Results in FIG. 4B, which show the corresponding FUDR to lipid ratio (+/−standard deviation) plotted at the specified times, indicates that FUDR retention is significantly decreased in vivo as the cholesterol content is increased from 5 to 20 mole %, with 10 mole % cholesterol showing better retention than 5 mole %. Together, these results demonstrate that DSPC/DSPG/Chol liposomes containing both FUDR and CPT-11 display optimal CPT-11 plasma levels when approximately 15 mole % cholesterol is used and optimal FUDR plasma concentrations when approximately 10 mole % cholesterol is used. Therefore, in order to co-formulate DSPC/DSPG liposomes for enhanced delivery of high levels of FUDR and CPT-11 to tumor sites, it appears that approximately 10-15 mole % of cholesterol is necessary for maintaining suitable drug retention and sustained drug release of each FUDR and CPT-11.

### Example 4

### Combinations of CPT-11 and FUDR Demonstrate Both Non-Antagonistic and Antagonistic Effects

Many combinations of two or more drugs have the ability to exhibit synergistic effects. Similarly, combinations of the same two or more drugs may show additive or antagonistic interactions depending upon the concentration of drug(s) used. In order to identify ratios of water-soluble camptothecins to fluoropyrimidines that are synergistic, various combinations of FUDR to CPT-11 were tested for their cytotoxic effects in vitro. More specifically, combinations that demonstrate synergy over a broad drug concentration range were identified.

Measuring additive, synergistic or antagonistic effects was performed using FUDR/CPT-11 at 10:1, 5:1, 1:1, 1:5 and 1:10 mole ratios in HT-29 human colorectal adenocarcinoma, H460 human large cell carcinoma, and HCT-116 human colorectal carcinoma cells. The standard tetrazolium-based colorimetric MTT viability assay protocol (Mosmann, et al., J. Immunol Methods (1983) 65(1-2):55-63) was utilized to determine the readout for the fraction of cells affected. Briefly, viable cells reduce the tetrazolium salt, 3-(4,5-dimethylthiazol-2-yl)-2,5-diphenyl-2H tetrazolium bromide (MTT) to a blue formazan which can be read spectrophotometrically. Cells, such as the HT-29 cell line used here, are grown in 25 cm² flasks and passaged (passage number <20), resuspended in fresh cell culture medium and added into 96-well cell culture plates at a concentration of 1000 cells per well in 100 μL per well. The cells are then allowed to incubate for 24 hours at 37° C., 5% CO₂. Single-cell preparations such as these may also be prepared from patient tumors or biopsies by homogenizing the tissue with known techniques. The following day, serial drug dilutions are prepared in 12-well cell culture plates. The agents, previously prepared in various solutions, are diluted in fresh cell culture media. Agents are administered to the appropriate or specified wells for single agents (20 μL) and at specific fixed ratio dual agent combinations (increments of 20 μL) using a Latin square design or “checkerboard” dilution method. The total well volumes are made up to 200 μL with fresh media. The drug exposure is for a duration of 72 hours.

Following drug exposure, MTT reagent (1 mg/mL in RPMI) is added to each well at a volume of 50 μL per well and incubated for 3-4 hours. The well contents are then aspirated and 150 μL of dimethylsulfoxide (DMSO) is added to each well to disrupt the cells and to solubilize the formazan precipitate within the cells. The 96-well plates are shaken on a plate shaker, and read on a microplate spectrophotometer set at a wavelength of 570 nm. The optical density (OD) readings are recorded and the OD values of the blank wells (containing media alone) are subtracted from all the wells containing cells. The cell survival following exposure to agents is based as a percentage of the control wells (cells not exposed to drug). All wells are performed in triplicate and mean values are calculated.

A combination index was determined for each FUDR/CPT-11 dose using Calcusyn which is based on Chou and Talalay's theory of dose-effect analysis, in which a “median-effect equation” has been used to calculate a number of biochemical equations that are extensively used in the art. Derivations of this equation have given rise to higher order equations such as those used to calculate Combination Index (CI). As mentioned previously, CI can be used to determine if combinations of more than one drug and various ratios of each combination are antagonistic (CI>1.1), additive (0.9≦CI≧1.1) or synergistic (CI<0.9). CI plots are typically illustrated with CI representing the y-axis versus the proportion of cells affected, or fraction affected (fₐ), on the x-axis. The data in FIG. 5, plotted as CI versus the fraction of HT-29 cells affected, clearly illustrate that particular combinations of FUDR and CPT-11 are antagonistic while others are synergistic or additive. At a FUDR:CPT-11 ratio of 5:1 or 1:1, synergy is observed over the entire range of fraction affected values (0.2 to 0.8). This demonstrates that a 5:1 or 1:1 ratio is synergistic independent of the concentration of each drug used. However, a 10:1 ratio is non-antagonistic at fₐ values below 0.76 and a 1:5 mole ratio of FUDR/CPT-11 is non-antagonistic at fₐ values less than 0.62, thus demonstrating that the synergy observed with these ratios are dependent upon the concentration of drugs employed. A FUDR:CPT-11 1:10 ratio is antagonistic over a substantial range of fₐ values (more than 50%).

A further representation used to identify an optimal drug ratio is to prepare a relative synergy plot, FIG. 5D. This method of analysis is useful for identifying a common synergistic drug:drug ratio from many cell types. FIG. 5D consists of compiled data sets at 1:5, 1:1, and 1:10 molar ratios from various cell types, as indicated on the ordinate axis. The relative synergy values shown on the abscissa are computed CI values obtained from CalcuSyn that have been normalized to zero by subtracting 1 from the original CI value, i.e., CI values of 0, 1, and 2 are equivalent to relative synergy values of −1, 0, and 1, respectively. This data format more clearly illustrates that at the evaluated ratio in some cell types synergy is observed (bars projecting to the left) while others are antagonistic (bars projecting to the right). In addition this method of analysis is beneficial for identifying tumor types that display similar synergistic responses at a 1:1 ratio, i.e., the similar levels of relative synergy observed with the colorectal cell lines, colon-26, HCT-116, HT-29 and LS180. Based on these results, a mole ratio of 1:1 FUDR:CPT-11 was selected for pharmacokinetic and efficacy studies described in the following examples as this ratio demonstrated synergistic effects that were independent of concentration. This is important as the concentration of drugs in likely to change in vivo following administration.

### Example 5

### Maintaining Ratios of Drugs In Vivo

To determine if a synergistic ratio of a water-soluble camptothecin and a fluoropyrimidine could be maintained in dual-loaded liposomes in vivo, DSPC/DSPG/Chol liposomes containing encapsulated FUDR and CPT-11 were administered intravenously to mice and the plasma drug/drug ratio was monitored over time.

FUDR and CPT-11 were formulated into DSPC/DSPG/Chol (70:20:10 mole ratio) liposomes as previously described at a 1:1 mole ratio identified in Example 4 to be synergistic. Lipid films were hydrated in a solution consisting of 100 mM Cu(gluconate)₂, 220 mM TEA, pH 7.4 and 30 mg/mL of FUDR (with trace amounts of ³H-FUDR). The resulting MLVs were extruded at 70° C. Subsequently, the liposomes were buffer exchanged into SHE, pH 7.4, by tangential flow dialysis, thus removing any unencapsulated FUDR and Cu(gluconate)₂.

CPT-11 was added to these liposomes at an initial CPT-11 to lipid ratio of 0.12:1. Loading of CPT-11 into the liposomes was facilitated by incubating the samples at 50° C. for 10 minutes. After loading, the samples were exchanged into saline (0.9% Sodium Chloride Injection, USP; pH 5.5, Baxter), by tangential flow dialysis to remove EDTA and unencapsulated drug. The extent of CPT-11 loading was measured using absorbance at 370 nm against a standard curve. FUDR and lipid levels were measured using liquid scintillation.

The preparations were injected intravenously via the tail vein into either CD-11 or SCID-Rag2M mice. Doses of the liposomal formulations were 8.38 mg/kg of FUDR and 20 mg/kg of CPT-11. For CD-1 mice, a comparison of the dual-loaded liposomes with a free drug cocktail of FUDR and CPT-11 at a 1:1 was also performed. The free drug cocktail was diluted in saline prior to administration to mice. At the indicated time points after intravenous administration, blood was collected by cardiac puncture (3 mice per time point) and placed into EDTA coated microcontainers. The samples were centrifuged to separate plasma and plasma was transferred to another tube. Liquid scintillation counting was used to quantitate radiolabeled lipid and FUDR in the plasma. CPT-11 plasma levels were quantified with HPLC.

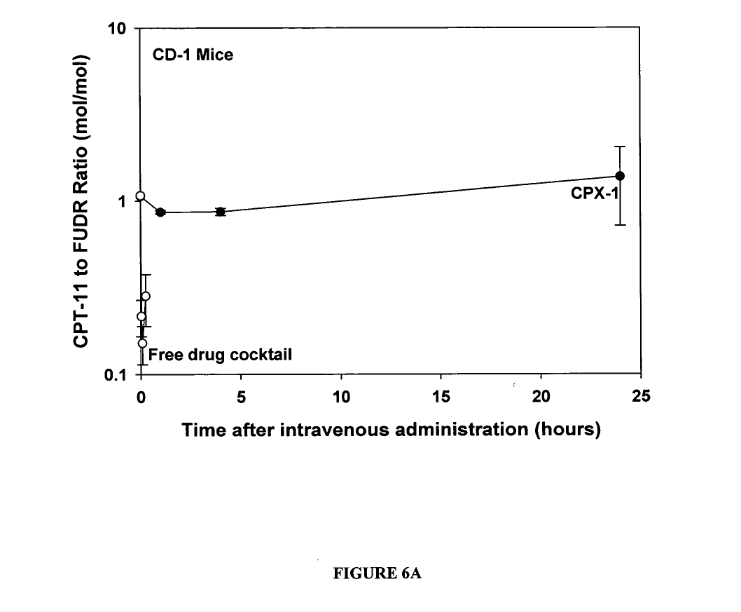

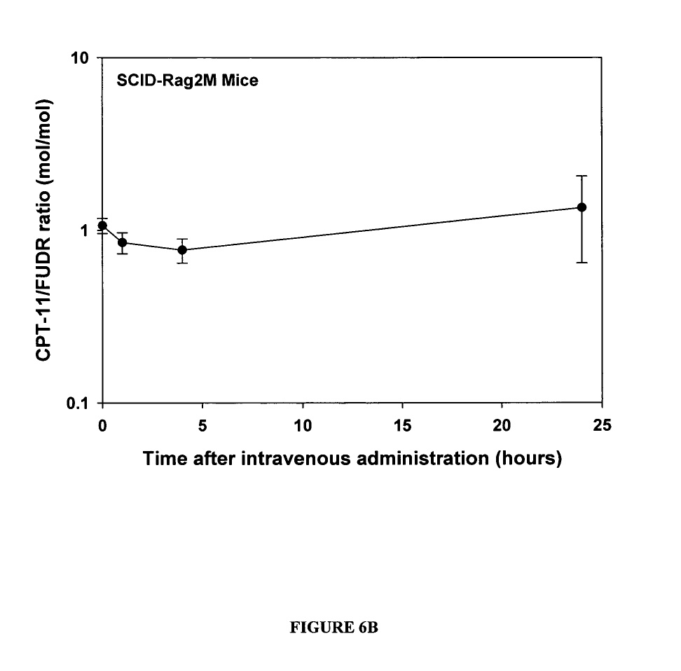

FIG. 6A shows that plasma levels of FUDR and CPT-11 were effectively maintained at a 1:1 mole ratio as plasma levels of FUDR were roughly equal to that of CPT-11 at various time points after intravenous administration to CD-1 mice when they were delivered in the above-described liposomes. In contrast, the free drug cocktail of FUDR/CPT-11 rapidly changed from the initial 1:1 mole ratio after administration. FIG. 6B similarly shows that plasma levels of FUDR and CPT-11 were maintained at a 1:1 mole ratio over time after administration to SCID-Rag2M mice. Data points represent the molar ratios of CPT-11/FUDR determined in plasma (+/−standard deviation) at the specified time points. These results clearly demonstrate that formulation of FUDR and CPT-11 into liposomes of the invention effectively maintains a synergistic ratio of these drugs in the plasma over time whereas a free drug cocktail results in a rapid and uncontrolled change of the drug/drug ratio from the optimum ratio required for synergistic killing of cancer cells. Therefore, it appears that for adequate in vivo delivery of a desired ratio of FUDR and CPT-11, it is ideal to formulate these drugs into lipid-based delivery vehicles.

### Example 6

### Simultaneous Encapsulation of CPT-11 and FUDR

An alternative method for the co-encapsulation of CPT-11 and FUDR was developed that allows for the simultaneous loading of both drugs. A lipid film was prepared by dissolving DSPC and cholesterol separately at 50 mg/ml in chloroform, and DSPG at 25 mg/ml in chloroform/methanol/water (50/10/1). The lipids were then combined together in the appropriate amounts to generate a DSPC/DSPG/Chol (70:20:10 mole ratio) mixture and then labeled with ¹⁴C-CHE. Solvent was removed under a stream of N₂ gas until very little solvent remained. The lipid film was then left under vacuum overnight on a vacuum pump to remove any residual solvent. The lipid film was rehydrated in 4 mL 100 mM Cu(gluconate)₂, 220 mM TEA, pH 7.4 at 70° C. and the resulting MLVs were extruded at 70° C. through two stacked 100 nm filters for a total of eight passes. Aliquots were taken before extrusion to determine the specific activity of the lipid preparation. The mean diameter of each sample was determined by QELS (quasi-elastic light scattering) analysis to be 100 nm+/−20 nm. The liposomes were then buffer exchanged into 150 mM NaCl, 20 mM Hepes, pH 7.4 (HBS) using a hand-held tangential flow column.

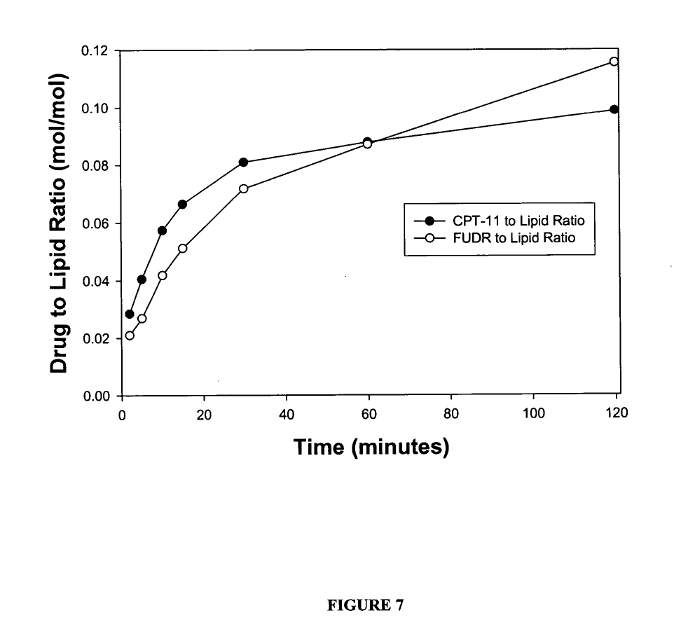

The CPT-11/FUDR uptake experiments were performed as follows: 25 μmoles of lipid, 3 μmoles of CPT-11 and 60 μmoles of FUDR (containing some ³H-FUDR) were incubated separately at 50° C. and then combined for a total volume of 500 μl. At various time points after mixing, an aliquot was removed and the external liposomal solution was exchanged for saline using 1 mL Sephadex G-50 columns. A drug to lipid ratio for the eluant was generated using dual label liquid scintillation counting to determine lipid and FUDR concentrations. CPT-11 was quantified by its absorbance at 370 nm against a standard curve. The conditions of the CPT-11 UV assay are as follows: A 100 μL aliquot of each liposomal sample was solubilized in 100 μL of 10% Triton X-100+800 μL 50 mM trisodium citrate/citric acid, 15 mM EDTA, pH 5.5 and then heated to 100° C. using boiling water until cloudy. Samples were cooled to room temperature before absorbance readings were taken. The simultaneous loading of CPT-11 and FUDR is shown in FIG. 7.

### Example 7

### Liposome-Encapsulated Drugs Demonstrate Enhanced Efficacy Over Synergistic Drug Cocktails

Many therapies today, particularly combination chemotherapies, rely on administration of free drug cocktails. The investigators here wanted to establish whether enhanced efficacy is observed in liposome-encapsulated drug combinations compared to free drug cocktails of the same combinations. The efficacies of dual-loaded liposomes was also compared to the therapeutic effects of each drug loaded separately into like liposomes.

DSPC/DSPG/Chol (70:20:10 mole ratio) liposomes co-encapsulated with FUDR and CPT-11 at a synergistic mole ratio of 1:1 were prepared as described in Example 1. DSPC/DSPG/Chol (70:20:10 mole ratio) liposomes containing either FUDR or CPT-11 were also prepared as in Example 1 except that only one drug was loaded per liposome using the respective loading methods previously mentioned. Lipid films were hydrated in 100 mM Cu(gluconate)₂, 220 mM TEA, pH 7.4. After hydration, the external liposome buffer was exchanged into SHE, pH 7.4; following CPT-11 loading, the external buffer was exchanged again into 0.9% saline.

Briefly, in order to perform tumor studies on mice, animals are inoculated with tumor cells which are then allowed to grow to sufficient size. Using a 28 g needle, mice are inoculated subcutaneously with 1-2×10⁶ tumor cells on day 0 (one inoculum/mouse) in a volume of 50 μL.

When tumors reach a defined size of approximately 100 mm³ to 200 mm³, either one-day prior to treatment or on the day of treatment, all tumors are measured. After selecting the appropriate tumor sizes, excluding tumors too small or large, the tumors are randomly distributed (n=6) and the mean tumor volume of the groups are determined. Mice are organized into appropriate treatment groups consisting of control and treatment groups such as for example, saline control, liposome control, positive control and various dilutions of test articles.

Mice are injected intravenously with the required volume of sample to administer the prescribed dose (10 μL/g as indicated) to the animals based on individual mouse weights.

Tumor growth measurements are monitored using vernier calipers beginning on the day of treatment. Tumor length measurements (mm) are made from the longest axis and width measurements (mm) will be perpendicular to this axis. From the length and width measurements tumor volumes (cm³) are calculated according to the equation (L×W²/2. Animal weights and in-life observations are collected at the time of tumor measurement.

All animals are observed at least once a day, more if deemed necessary, during the pre-treatment and treatment periods for mortality and morbidity. In particular, signs of ill health are based on body weight loss, change in appetite, rough coat, lack of grooming, behavioral changes such as altered gait, lethargy and gross manifestations of stress. Should signs of severe toxicity or tumor-related illness be seen, the animals are euthanized (CO₂ asphyxiation) and a necropsy is performed to assess other signs of toxicity. Moribund animals are terminated for humane reasons and the decision to terminate is at the discretion of the Study Director/Manager and the Animal Care Technician. Any and all of these findings will be recorded as raw data and the time of death will be logged as the following day.

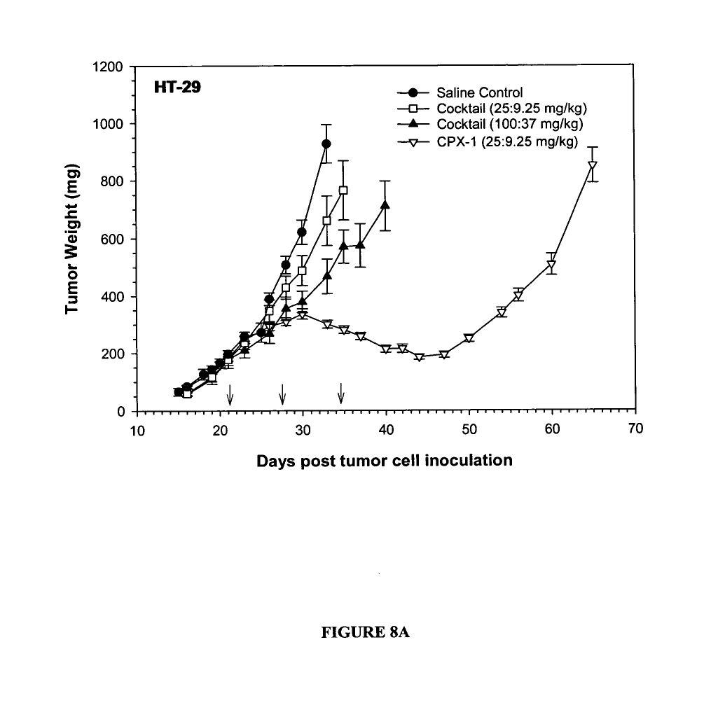

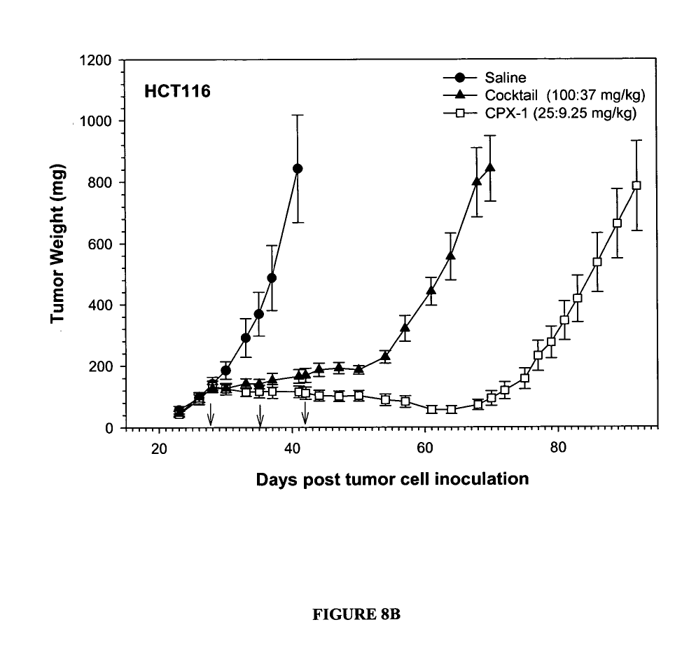

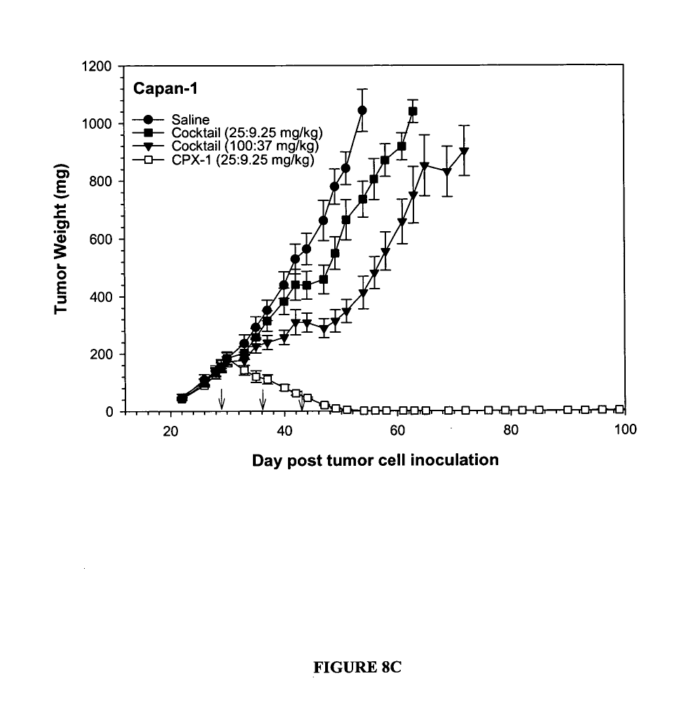

In this study, performed as described above, efficacy experiments were carried out in female SCID-Rag2M mice that had been inoculated subcutaneously in the flank with either 2×10⁶ human HT-29 or HCT 116 colon adenocarcinomas , 2×10⁶ human Capan-1 pancreatic tumor cells or 1×10⁶ murine Colon-26 adenocarcinoma. Xenograft tumors were allowed to grow until they measured to be approximately 150 mg (150 mm³) in size, and the murine tumors were grown to approximately 100 mg (100 mm³), at which time the indicated formulations were injected. Tumor growth was determined by direct caliper measurements. Mice were treated with a multiple dosing schedule (arrows in FIGS. 8A, 8B and 8C indicate the days of treatment) of saline, free drug cocktail at a 1:1 mole ratio, individual free agents, a liposomal formulation of CPT-11:FUDR at a 1:1, 1:10 and 10:1 mole ratio, and FUDR and CPT-11 were also administered individually in liposomes, in order to compare the efficacy of single drug-loaded liposomes (“liposomal FUDR” and “liposomal CPT-11”) with dual-loaded liposomes (“liposomal CPT-11:FUDR”). Antitumor activity was quantitated by calculating the percent tumor growth delay and log cell kill. Percent tumor growth delay is defined as the time in days taken for treated tumors to reach the specified evaluation size as a percent of control (T−C/C×100) where T is the treatment tumors and C is the control tumors in days. Log cell kill is an estimate of the number of log₁₀ units of cells killed at the end of treatment defined as [T−C/(3.32)(Td)] where T−C is the treatment induced delay for tumors to reach a specified evaluation size and Td is the tumor doubling time in days. A log cell kill of 0 indicates that the cell population at the end of treatment is the same as it was at the start of treatment. A log cell kill of +6 indicates a 99.9999% reduction in the cell population.

Results presented in FIG. 8A show that administration of liposomal CPT-11:FUDR encapsulated in DSPC/DSPG/Chol (70:20:10) liposome at a 1:1 mole ratio at a dose of 25:9.25 mg/kg (corresponding lipid dose of 278 mg/kg) provided significantly better therapeutic activity (reduced human colorectal HT-29 tumor size) when compared to animals treated with either the free drug cocktail dose matched at 25:9.25 mg/kg and at the maximum tolerated dose (MTD) of 100:37 mg/kg or saline. The graph in FIG. 8B illustrates that administration of liposomal CPT-11:FUDR encapsulated in DSPC/DSPG/Chol (70:20:10) liposome at a 1:1 mole ratio at a dose of 25:9.25 mg/kg (corresponding lipid dose of 278 mg/kg) provided significantly better therapeutic activity (reduced human colorectal HCT116 tumor size) when compared to animals treated with the free drug cocktail at the maximum tolerated dose (MTD) of 100:37 mg/kg or saline. Similar to the results in FIG. 8A, the graph in FIG. 8C demonstrates that administration of liposomal CPT-11:FUDR encapsulated in DSPC/DSPG/Chol (70:20:10) liposome at a 1:1 mole ratio reduced tumor size, in mice with tumors derived from human Capan-1 pancreatic tumor cells, to a larger degree than either a free drug cocktail of CPT-11:FUDR or saline. Data points represent mean tumor size +/−standard error of the mean (SEM). Together, these results strongly indicate that encapsulation of fluoropyrimidines and water-soluble camptothecins into well-designed delivery vehicles is required to achieve optimal therapeutic activity.

Table 1A below contains tabulated quantitative anti-tumor efficacy data in the human HT-29 colorectal xenograft model followed by administration of liposomal CPT-11:FUDR at a 1:1 molar and 10:1 molar ratio, liposomal FUDR, liposomal CPT-11, free drug cocktail CPT-11:FUDR at a 1:1 molar ratio and free agent CPT-11 and free FUDR.

TABLE 1A
Quantitative Analysis of HT-29 Antitumor Activity
for Treatment Groups
(Q7D × 3 treatment schedule)

| | | Tumor | Percent tumor | |
|---|---|---|---|---|
| | Dose | growth delay | growth delay | Log cell |
| Treatment | (mg/kg) | (days) | [(T − C ÷ C) × 100] | kill* |
| Free Flox | 250 | 2 | 8% | 0.09 |
| Free Irino | 100 | 2 | 8% | 0.09 |
| Cocktail | 100:37 | 5 | 19% | 0.23 |
| L-Flox | 18.5 | 3 | 12% | 0.14 |
| L-Irino | 50 | 33 | 127% | 1.51 |
| Antagonistic | 50:1.85 | 32 | 123% | 1.48 |
| CPX-1 | 50:18.5 | 37 | 142% | 1.71 |

*Log cell kill = [T − C/(3.32)(T_{d})] where T − C is the treatment induced delay for tumors to reach 400 mg (in days) and T_{d} is the tumor doubling time in days. Days for control tumors to reach 400 mg = 26 days.

Results presented in Table 1A illustrate a quantitative analysis of HT-29 antitumor activity for the indicated treatment groups. After administration of liposomal FUDR (L-Flox) at a dose of 18.5 mg/kg there was a tumor growth delay of only 12% and a log cell kill of 0.14. Liposomal CPT-11 (L-Irino) at a dose of 50 mg/kg was highly active with a tumor growth delay of 127% and a log cell kill of 1.51. The administration of liposomal CPT-11:FUDR encapsulated in DSPC/DSPG/Chol (70:20:10) liposome at a 1:1 mole ratio dosed at 50:18.5 mg/kg (corresponding lipid dose 556 mg/kg) (CPX-1) was most efficacious with a tumor growth delay of 142% and a log cell kill of 1.71. Liposomal CPT-11:FUDR encapsulated in DSPC/DSPG/Chol (70:20:10) liposome at an antagonistic 10:1 mole ratio dosed at 50:1.85 mg/kg (Antagonistic) was less efficacious than the 1:1 mole ratio with a tumor growth delay of 123% and a log cell kill of 1.48. The free drug cocktail at the maximum tolerated dose (MTD) of 100:37 mg/kg exhibited a 19% tumor growth delay and a log cell kill of 0.23. Both free CPT-11 and free FUDR dosed at 100 and 250 mg/kg respectively had a 8% tumor growth delay and a log cell kill of 0.09.

Table 1B contains tabulated quantitative anti-tumor efficacy data in the human Capan-1 pancreatic xenograft model followed by administration of liposomal CPT-11:FUDR at a 1:1 molar and 1:10 molar ratio, liposomal FUDR, liposomal CPT-11 and free agent CPT-11 and free FUDR.

TABLE 1B
Quantitative Analysis of Capan-1 Antitumor
Activity for Treatment Groups
(Q7D × 3 treatment schedule)

| | | Tumor | Percent tumor | |
|---|---|---|---|---|
| | Dose | growth delay | growth delay | Log |
| Treatment | (mg/kg) | (days) | [(T − C ÷ C) × 100] | cell kill* |
| Free Irino | 100 | 15 | 35% | 0.65 |
| Free Flox | 250 | 10 | 23% | 0.43 |
| L-Irino | 25 | 34 | 79% | 1.46 |
| L-Flox | 9.25 | 7 | 16% | 0.30 |
| L-Flox | 18.5 | 10 | 23% | 0.43 |
| Antagonistic | 5:18.5 | 10 | 23% | 0.43 |
| CPX-1 | 25:9.25 | 42 | 98% | 1.98** |

*Log cell kill = [T − C/(3.32)(T_{d})] where T − C is the treatment induced delay for tumors to reach 500 mg (in days) and T_{d} is the tumor doubling time in days. Days for control tumors to reach 500 mg = 43 days.
**Statistically different (p < 0.05) from all other groups using Student-Newman-Keuls analysis

Similar to the results in Table 1A, results tabulated in Table 1B illustrate that liposomal CPT-11:FUDR exhibited superior anti-tumor activity in the human Capan-1 pancreatic xenograft model at a dose of 25:9.25 mg/kg (corresponding lipid dose 278 mg/kg) (CPX-1) with a tumor growth delay of 98% and a log cell kill of 1.98, which was shown to be statistically significant in comparison to liposomal CPT-11 (25 mg/kg) (L-Irino) and liposomal FUDR (9.25 mg/kg) (L-Flox) that exhibited tumor growth delays of 79% and 16% and log cell kills of 1.46 and 0.30, respectively.

Table 1C contains tabulated quantitative anti-tumor efficacy data in the murine Colon-26 model followed by administration of liposomal CPT-11:FUDR at a 1:1 molar ratio, liposomal FUDR, and liposomal CPT-11.

TABLE 1C
Quantitative Analysis of Colon-26 Antitumor
Activity for Treatment Groups
(Q4D × 3 treatment schedule)

| | | Tumor | Percent tumor | |
|---|---|---|---|---|
| | Dose | growth delay | growth delay | |
| Treatment | (mg/kg) | (days) | [(T − C ÷ C) × 100] | Log cell kill* |
| L-Flox | 7.4 | 2.90 | 14.8% | 0.69 |
| L-Irino | 20 | 2.73 | 13.9% | 0.65 |
| CPX-1 | 20:7.4 | 10.40 | 53.06% | 2.47** |

*Log cell kill = [T − C/(3.32)(Td)] where T − C is the treatment induced delay for tumors to reach 500 mg (in days) and Td is the tumor doubling time in days. Days for control tumors to reach 500 mg = 19 days.
**Statistically different (p < 0.05) from all other groups using Student-Newman-Keuls analysis

Table 1C illustrates that liposomal CPT-11:FUDR exhibited superior anti-tumor activity in the murine Colon-26 colorectal model at a dose of 20:7.4 mg/kg (corresponding lipid dose 160 mg/kg) (CPX-1) with a tumor growth delay of 53% and a log cell kill of 2.47. Liposomal CPT-11 (20 mg/kg) (L-Irino) and liposomal FUDR (7.4 mg/kg) (L-Flox) resulted in tumor growth delays of 13.9% and 14.8% and log cell kills of 0.65 and 0.69, respectively. Both the single-loaded liposomes were more active than the saline control, and as expected, the liposomal CPT-11:FUDR was the most efficacious showing a reduction in tumor size that was statistically significant compared to liposomal CPT-11 and liposomal FUDR.

We claim:

1. A composition that comprises liposomes stably associated with at least one water-soluble camptothecin and at least one fluoropyrimidine at a camptothecin-to-fluoropyrimidine mole ratio that has a desired cytotoxic, cytostatic or biologic effect to relevant cells or tumor cell homogenates.

2. The composition of claim 1 wherein the desired cytotoxic, cytostatic or biologic effect to relevant cells or tumor cell homogenates is non-antagonistic.

3. The composition of claim 1 which further includes leucovorin sufficient to stabilize said fluoropyrimidine.

4. The composition of claim 2 which further includes leucovorin sufficient to stabilize said fluoropyrimidine.

5. The composition of claim 1 wherein the water-soluble camptothecin is irinotecan (CPT-11), topotecan, 9-aminocamptothecin or lurtotecan.

6. The composition of claim 1 wherein the water-soluble camptothecin is a hydrophilic salt of a water-insoluble camptothecin.

7. The composition of claim 2 wherein the water-soluble camptothecin is irinotecan (CPT-11) or topotecan.

8. The composition of claim 1 wherein the fluoropyrimidine is floxuridine, fluorouracil or UFT (tegafur/uracil).

9. The composition of claim 1 wherein said liposomes comprise a phosphatidylcholine-containing lipid.

10. The composition of claim 9 wherein said phosphatidylcholine-containing lipid is DSPC or DAPC.

11. The composition of claim 1 wherein said liposomes comprise a phosphatidylglycerol or a phosphatidylinositol.

12. The composition of claim 11 wherein the phosphatidyl glycerol is DSPG or DMPG.

13. The composition of claim 1 wherein said liposomes comprise a sterol.

14. The composition of claim 13 wherein said sterol is cholesterol.

15. The composition of claim 14 wherein said cholesterol is present at less than 20 mol%.

16. The composition of claim 1 wherein said liposomes comprise a metal ion solution.

17. The composition of claim 16 wherein said metal ion is copper.

18. The composition of claim 17 wherein said metal ion solution is Cu(gluconate)₂ or CuSO₄.

19. The composition of claim 1 wherein said water-soluble camptothecin and fluoropyrimidine are co-encapsulated.

20. The composition of claim 1 wherein said water-soluble camptothecin is irinotecan or topotecan and said fluoropyrimidine is floxuridine or 5-FU.

21. The composition of claim 20 wherein said liposomes comprise DSPC.

22. The composition of claim 20 wherein said liposomes comprise DSPG.

23. The composition of claim 20 wherein said liposomes comprise cholesterol.

24. The composition of claim 20 wherein said liposomes comprise Cu(gluconate)₂ or CuSO₄.

25. The composition of claim 20 wherein said liposomes comprise triethanolamine (TEA).

26. The composition of claim 1 which, when administered to a subject, provides a therapeutic activity greater than that which is obtained when said water-soluble camptothecin and said fluoropyrimidine are administered in the same ratio but not stably associated with liposomes.

27. The composition of claim 1 wherein the composition comprises a third agent.

28. A method to prepare a composition comprising liposomes, said liposomes having stably associated therewith at least one water-soluble camptothecin and one fluoropyrimidine in a mole ratio which is non-antagonistic, which method comprises
a) determining in a relevant cell culture assay, cell-free assay or tumor cell homogenate for biological activity a mole ratio of said water-soluble camptothecin and fluoropyrimidine agents which is non-antagonistic over at least 5% of the concentration range over which greater than 1% of cells are affected (fₐ>0.01) by said ratio of agents, and
b) encapsulating within said liposomes a mole ratio of water-soluble camptothecin-to-fluoropyrimidine determined to be non-antagonistic in step a).

29. A method to treat a disease condition in a subject which method comprises administering to a subject in need of such treatment a therapeutically effective amount of the composition of claim 1.

30. The method of claim 29 which further comprises administering leucovorin to said subject.

31. The method of claim 29 wherein the subject is a human.

32. The method of claim 29 wherein the subject is a non-human mammal or avian.

33. A method to deliver a therapeutically effective amount of a fluoropyrimidine/water-soluble camptothecin drug combination by administering a fluoropyrimidine stably associated with a first delivery vehicle and a water-soluble camptothecin stably associated with a second delivery vehicle wherein the ratio of the fluoropyrimidine and the water-soluble camptothecin administered is non-antagonistic.
